(19) United States
(12) Patent Application Publication
(10) Pub. No.: US 2019/0183662 A9
(43) Pub. Date: Jun. 20, 2019

(54) TRANSFEMORAL LEVEL INTERFACE SYSTEM USING COMPLIANT MEMBERS

(71) Applicant: James Jay Martin, Oklahoma City, OK (US)

(72) Inventor: James Jay Martin, Oklahoma City, OK (US)

(21) Appl. No.: 15/690,888

(22) Filed: Aug. 30, 2017

Related U.S. Application Data
(63) Continuation of application No. 14/708,274, filed on May 10, 2015, now abandoned.
(60) Provisional application No. 61/998,569, filed on Jul. 1, 2014.

(51) Int. Cl. A61F 2/60; A61F 2/80

(52) U.S. Cl. CPC A61F 2/60; A61F 2002/5016; A61F 2002/607; A61F 2/80

(57) ABSTRACT

A transfemoral prosthetic level socket system for a user's lower limb comprising modular socket components fitted to the individual user's residual limb having a mounting point for an attachment, at least one compliant member attached to at least one stabilizing unit, and at least one second compliant member attached to at least one stabilizing unit wherein the first compliant member and the second compliant member work in cooperation with the stabilizing unit(s) to control bone position and support the limb within the interface.

### CROSS-REFERENCE TO RELATED APPLICATIONS

The present application is a continuation of U.S. Ser. No. 14/708,274, filed May 10, 2015, currently pending, which priority is claimed from provisional application U.S. Ser. No. 61/998,569 filed Jul. 1, 2014 and each application is incorporated by reference herein.

### BACKGROUND OF THE INVENTION

### 1. Field of the Invention

The present invention relates generally to a prosthesis for transfemoral or knee disarticulation levels of amputation. More particularly, the present invention is a new and improved prosthetic socket apparatus and system utilizing less obtrusive trim lines, reduced weight, increased flexibility, and increased control of the prosthesis, and general increased comfort to the user.

### 2. Background of the Invention

It is estimated that by the year 2050, the number of amputees will double to over 3.6 Million. American population health concerns are strongly correlated to the weight and age of the individual. The American population and its health patterns show an increase in all high-risk areas as related to dysvascular disease, a leading cause of both stroke and amputation. In the United States, 97% of dysvascular related amputations involve the lower limb.

The aging baby-boomer population is just entering the age where vascular insufficiencies tend to drastically increase, thus, a large influx of stroke patients and amputee patients will be entering the U.S. market alone in the coming few years.

The prosthetics market has advanced significantly in recent years—now utilizing many new advanced components such as computer controlled knees and feet. However, the socket interfaces are minimally different from what was being used 20 to 30 years ago. In fact, many of the socket interfaces that are still considered state-of-the-art were originally developed between the 1960's and 1990's, with only minor advancements in materials and suspension methods since then. The core socket interface designs have gone largely unchanged.

There are numerous prosthetic and orthotic companies that provide components for conventional interface designs—ranging from various embodiments of gel liners and suspension aids for prosthetics users. In each of these, the core interface approaches used are antiquated and in desperate need of more advanced methods of how to fit prosthetic devices.

The current state-of-the-art fails to truly accommodate for limb volume changes, has narrow transitions from high force to no force resulting in rubbing and discomfort, and creates a volatile skin environment that is hot and sweaty. Conventional transfemoral socket shapes hydrostatically compress the residual limb and its tissues into a tight-fitting pre-determined bucket shape, often referred to as a “socket”. The lineage of transfemoral design typically includes compressing the soft tissue around the underlying muscle-channels and locking around the tuberosity/ramus in various orientations. Conventional socket shapes remain static in size and shape. As the human limb changes in shape and volume, the “key in a keyhole” fit is lost.

The tight fitting nature of conventional interfaces leads to rubbing at the trim lines. At this level, the tissue compression goes from high force to no force over a narrow band, resulting in shear forces on the fragile tissue. The use of rigid or semi-rigid materials in conventional socket interfaces compounds this problem. There is a need for softer, more dynamic materials to be used at transition areas, allowing for compliant transition zones.

The hostile interface environments of conventional prosthetics are hot, full of constant perspiration, lack breathability, and are heavy. As such, many users suffer from various skin conditions including: skin breakdown, pressure sores, heat rash, abrasions, chaffing, dryness, folliculitis, dermatitis, ulcers, eczema, psoriasis, and dry skin. Prosthetics users are desperately searching for better solutions to the many outstanding problems associated with conventional socket interfaces.

The socket interface is by far the most important component of a successful prosthetic or orthotic device. While advanced computer controlled joints are incredibly beneficial for enhancing functional performance, if the socket interface is not stable and comfortable, the high-tech components have little advantage.

Orthotics and prosthetics users are desperately searching for better solutions to the many outstanding problems associated with conventional socket interfaces. Much of the work in advancing the socket comfort has been focused around new materials and suspension capabilities of gel liners, and lighter weight carbon fiber materials. However, the inherent socket interface designs themselves have seen very little change in how they are fit to the patient.

For prosthetics users, the gel liners are a soft padding that resides between the users' limb and the hard socket interface. While these do provide more cushion, they do not address volume change issues, and tend to be very hot, full of perspiration, and make for a hostile environment for the skin. The application of a more advanced compliant-based socket interface approach will greatly enhance the functionality and the daily living for the end user.

Weight Reduction

Prosthetics devices are a significant weight hanging off the body. The Compliant Force Distribution socket interface designs, as disclosed herein, may use compliant materials within its surface area, versus thermoplastic and carbon fiber rigid encapsulated sockets, which are inherently heavier. Thus, the compliant-based socket design may offer a reduction in weight of over 50%.

Keeping the actual weight of prosthetic devices as low as possible is critically important for the efficiency and comfort of the user, but just as important is the perceived weight. The more intrinsic bone motion within the residual limb due to soft tissue pliability in a conventional hydrostatic socket fit results in a significant loss of biomechanical efficiency, and an increase in the perceived weight of the device.

The dynamic Compliant Force Distribution socket interface designs provide for an inherently greater biomechanical lock around the underlying bony structure, providing for a greater one to one connectivity between the user's body and the device—further decreasing perceived weight.

Point Pressures and Force Distribution

One of the main areas causing abrasions within a conventional prosthetic or orthotic socket interface is the trim lines. The trim lines are where there is a rapid transition from high pressure to no pressure. Because the conventional socket interface does not change in shape with the dynamic underlying body, the user's skin often rubs at the trim lines, causing abrasions. Conventionally used padded straps for instance are around 1″-2″ wide and are typically pulled tight to provide for stability. The skin under the straps is highly compressed, and the skin just outside of the straps is not. This narrow area of high force to no force creates a shear point. The same issue is found at trim lines of the socket.

The compliant socket designs however eliminate the conventional trim-line transitions, and are replaced with a broad compliant fabric, allowing for a significantly broader distribution of pressures, and a very gradual transition from high forces to no forces.

No matter how much “padding” is used with conventional straps or trimlines, the forces remain distributed within a small surface area. Compliant Force Distribution provides a more gradual transition of forces at the edges, and equalizes the amount of force per square inch within the load bearing areas.

Sensor Integration

The Compliant Force Distribution socket interface technology has been successfully applied to high-level upper extremity amputees. In conventional upper extremity socket designs, the electrodes tend to gap away from the body, as they would typically be integrated into a rigid or semi-rigid socket over a dynamic body. Compliant Force Distribution interface techniques instead maintain consistent electrode contact, as the sensors are “hammocked” directly to the user's limbs, with no loss of connection.

The future of socket designs for other levels of amputation, as well as for some orthotics users will incorporate various sensors, including myoelectric electrodes. Other than the Compliant Force Distribution Technology, there is not an elegant method of incorporating sensors and wires within a socket interface environment. These designs solve the outstanding issues that prevent the practical use of sensors and electrodes within the socket, as they are akin to a hammock and inherently hold the interface tightly against the users' limb—maintaining consistent contact.

Skin Environment

Conventional interface designs encompass the user's limb, creating a hot, moist environment that leads to a variety of skin issues including pressure sores, heat rash, abrasions, chaffing, dryness, folliculitis, dermatitis, ulcers, eczema, psoriasis, dryness, and skin breakdown.

The human skin is designed to breath. By design, the human skin perspires to cool itself, but instead, any perspiration within a conventional socket is trapped and a prosthetics user for instance typically can pour sweat out of their socket upon taking it off.

The Compliant Force Distribution interface designs however may be breathable, and allow for perspiration to escape, and the limb to remain cool, naturally.

Control and Stability

In transfemoral amputations for instance, the cut femur bone is no longer directly connected to the remaining leg. With a conventional hydrostatic socket design, the cut femur bone moves back and forth within the soft tissue of the residual limb, decreasing ambulation efficiency.

Randy Alley recently created the Hi-Fidelity socket to better lock the residual bone in a consistent position within the socket (U.S. Pat. No. 8,323,353). His work with the Hi-Fi demonstrates the ability to alter the biomechanical synchronization between the residual bone and prosthesis by changing how the socket interface is fit to the user.

Likewise, the Compliant Force Distribution design is able to replicate the lost biomechanical and neuromuscular connection between the limb and the user by better controlling underlying bone position within the socket, though through a radically different method. Just as importantly, it does so in a modular, breathable way that also accommodates for volume changes. Unlike Randy Alley's socket design, this approach captures the underlying bony structure in a much less aggressive and more compliant manner, making it easier to achieve more comfort. Instead of using aggressive compression zones that encircle the limb, this design more elegantly captures the contouring of the underlying anatomy to lock the bony structure in a desired orientation with respect to the prosthesis.

By more elegantly spreading the load over a broad surface area, our technology is not only reducing the amount of force per square inch, but more importantly, is providing a much more secure connectivity between the user and the device. A prosthetic or orthotic device is a mechanical or electro-mechanical extension from the body. Every amputee for instance gains what is called external physiological proprioception. This means that they are able to at least partially gain a proprioceptive sense of where the prosthesis is in space, in relation to their body. When they move their leg forward to take a step, they have a sense of where their leg position is in space, even though their prosthesis is not neurally connected to their brain. However, the more “wiggle room” there is between their residual limb skeletal system and the prosthesis, the less specificity they have of a true proprioceptive sense of their leg position.

For transfemoral amputees for instance, the femur bone section is cut at the amputation level and is no longer directly connected to the remaining leg. With a conventional hydrostatic socket design, when the transfemoral amputee kicks their leg forward to initiate the swing phase of gait, their femur bone moves back and forth within the soft tissue of their residual limb, and they lose much of the true proprioceptive sense of leg position.

Volume Accommodating—Dynamic Vs. Static

The human body is dynamic—yet the socket interfaces we use today are largely relatively static in their size and shape. Socket interfaces typically use a rigid or semi-rigid carbon fiber shell surrounding a semi-flexible thermoplastic inner layer. While this inner layer has some flexibility, its size and shape remain static, and cannot accommodate weight gain or loss. This leads to discomfort and degradation in functional performance of the user if their body changes in size and shape, and no longer perfectly matches the socket interface. Just a small body weight change of 5 lbs can significantly affect the socket interface fit. It is like wearing a shoe that is a couple sizes too big or too small—except that the effect is greatly compounded, as the limbs are not designed to bear the incredible ambulation forces, as the foot is designed to do.

The human body gains and loses volume due to hydration levels, foods we eat (for instance salty foods versus non-salty foods), exercise or lack thereof, eating habits, age related issues, and other diseases like diabetes and dysvascular disease.

If the socket does not perfectly fit, it leads to abrasions, rubbing, pressure on the cut end of the amputated bones, unwanted pressure on sensitive nerves, and overall discomfort. Most amputees and orthotics users experience socket interface discomfort from time to time. Socket interfaces tend to need to be replaced every 1 to 3 years due to body size and shape changes.

The Compliant Force Distribution technology inherently accommodates for size and shape changes in the user. These socket interface designs are compliant-based, versus a rigid or semi-rigid shape, therefore the socket interface can be quickly and easily user-adjusted in its tightness to provide a comfortable fit independent of weight gain or loss. As has been found with the Shoulder Disarticulation version of the Compliant Force Distribution socket interface design, the amputee is able to quickly and easily adjust the fit of their prosthetic device to ensure the most comfortable fit.

Conventional prosthetic socket designs could be more closely compared to wooden clog shoes, in that their size and shape do not adequately accommodate for the dynamic nature of the human body. Even if the wooden clog were to be perfectly contoured to the user's foot, its comfort would be limited, especially when the size and shape of the dynamic foot were to change. A compliant-based socket design would more closely resemble the Vibram five-fingered shoes, in that they are made of a soft dynamic material that perfectly contours to the user's foot, versus the foot matching to the shoe.

3. Description of the Known Prior Art

Conventionally used prosthetic interfaces remain as an anatomically contoured socket in which the residual limb fits within. This socket may be specifically tailored to the residual limb's size and shape, but it largely remains as a static size and shape. While some flexible materials may be incorporated, their flexibility is typically no more than minor amounts of give at their edges, and not true accommodation for the dynamic nature of the underlying body in which they are fit.

In recent years there have been a few attempts at improving lower extremity socket interface design, and overcome some of the outstanding issues surrounding them.

One such attempt developed by Randy Alley through the Hi-Fidelity (Hi-Fi) Interface (U.S. Pat. No. 8,323,353) design offers excellent locking around the underlying skeletal system, versus solely hydrostatic tissue loading as with conventional designs. However, the Hi-Fi system currently requires full customization for each user, and currently does not accommodate volume changes of the user any more so than other conventional transfemoral designs. It also uses an enclosed socket cavity in which the limb is fit, which does not fully address the environmental issues of limb encapsulation. While this design could in theory be transitioned to more of a modular approach, its design by nature would still require significant customization of the various components to fit appropriately to the user.

The Alley design requires a common distal connection point for each of the four vertical struts. The common distal connection point holds the vertical struts in a certain orientation about the limb for a transfemoral amputee, resulting in a locked-in socket shape. While the distal common connection point could become modular, once fit to the user, the position of the vertical struts is maintained in a consistent position, and is not quickly or easily adjustable for the patient.

It is important to offer a prosthetic interface design that is modularly adjustable by the end user, in real-time, so that they can match the socket to their limb, versus their limb having to match to the socket. In the Alley design, plastic shims may be used to adjust the static socket shape to the user, by placing them in between the static socket shape and the user's limb to take up space. In this invention however, our design can be modularly adjusted on the fly, allowing an end user to quickly and easily tighten or loosen to socket fit to match their desired comfort.

The Alley design uses struts that extend from the common distal connection point to the proximal brim of the socket. Our design instead uses floating force distribution anchors, to capture the long bone and conform it to the medial wall of the interface design. By using a floating design, the socket interface fully matches to the shape and size of the underlying limb with real-time adjustability, versus requiring a limb to match the shape of a customized socket fit to the patient, as in the Alley design.

Additionally, the Alley design uses the vertical struts as the structural element of the socket, and does not include any compliant members in the design. In our design, we instead make the force distribution anchors as floating, and not connected as a vertical structural element of the socket, in order to be compliant to match the compliant body, and then can integrate flexible fabric or flexible materials or adjustable connectors to span as the connectors. By doing such, this design can effectively leverage the long bone of the limb to become a structural element of the socket, versus solely relying on the carbon fiber struts to be the structural part of the design. Since this design captures the long bone with floating force distribution anchors, and manages the bone position to the stabilizing anchor, the long bone is controlled and its lock within the system assists in creating a structural element of system support.

The Alley design locks the long bone in a set position from all sides, as the vertical struts compress into the soft tissue circumferentially around the limb. This design instead pulls the long bone over to the stabilizing anchor, generating a more appropriate and controlled femoral angle.

According to the Alley patent, their design uses a limb encapsulated strut design where the struts are “appropriately contoured to a patient's residual limb” and “contains windows through which soft tissue can flow”. Our design instead is able to use force distribution anchors that can either be of an off-the-shelf shape, or can be dynamic in nature, not fully maintaining any particular shape, but rather match to the shape of the limb. In addition, our design does not have windows which the soft tissue can flow, but rather has un-encapsulated areas where there is not structure, and hence no windows.

Still further, Alley's design requires areas of specific isolated compression zones, whereas our disclosure provides broader areas of tissue stabilization, spreading the forces across more surface area than just isolated struts, and instead may utilize a combination of struts and compliant materials together to broaden the load bearing areas. In Alley's design, tissue is compressed such that the bone is locked in a position from all sides, due to the isolated tissue compression. Our design rather uses purposeful contouring around the bone such that a broader area of the limb is captured and controlled, and pulled toward the main anchor stabilizer along the medial aspect of the interface. Alley's design further calls for areas of the interface to be “enclosed or completely open provided there is minimal restriction to soft tissue flow”. Our design instead benefits from open areas where the soft tissue may be further controlled with compliant means, versus just relying on it to flow freely with minimal restriction.

Even further, Alley's patent calls for no less than 3 compression portions, while our approach utilizes just 2 pseudo-compression portions, where compliant means may be spanned in between. Our main anchor stabilizer may be sufficient in dimensions to not necessarily cause its own tissue compression in the same manner as other narrower struts would, as in Alley's design.

Still further, Alley's patent also calls for the struts to be of similar length as the long bone. In our design, the length may be modularly adjustable, and does not necessarily need to extend to the furthest distal end, or furthest proximal end, as our force distribution stabilizers are not directly connected to a common distal mounting point or proximal brim as in Alley's designs.

More recently Hurley's (LIM Innovations) (patent application number 2014/0135946 A1) introduced a modular version of Alley's Hi-Fi socket. Hurley's design accomplishes much the same as what was described by Alley, and functionally is equivalent how it fits to a residual limb, with the exception of being modular. Similar to Alley's design, the Hurley design requires significant customization, and a complex and time consuming fabrication and assembly process, and encapsulates the limb in a structure that surrounds the limb as a solid structural unit. While Hurley's design is modular in nature, the modularity of the design is suited well for a practitioner to modify it to a patient, but is not conducive for a patient to modify it on the fly in real-time. Hurley's design as well locks the long bone in a set position from all sides, as the vertical struts compress into the soft tissue circumferentially around the limb. The Hurley design shares most of the same disadvantages as Alley's design, as was discussed above, as they effectively have the same functions in how they fit about the underlying limb.

The recent RevoLimb design uses an adjustable Boa lacing system to slightly tighten or loosen various pads within the socket. This provides a step toward making a more accommodating socket though it still remains as a fully encapsulated limb environment, is complex to fabricate, requires significant customization, and is limited in its volume accommodation.

Cornell (U.S. Pat. No. 8,945,237) disclosed a transfemoral socket using a fabric spanned across one side of the frame, referred to as a sail. His disclosure spans the limb circumferentially, but fails to capture or manage the long bone. In his sail design, the limb is simply encapsulated with a combination of rigid frame, and flexible fabric, though the entire limb is encapsulated circumferentially giving a similar socket shape and effects as conventional socket designs. The main advantage that his sail material is that it provides more flexibility in sitting, and is adjustable. However, by not controlling the bone position within the socket, it fails to influence the biomechanical efficiencies while walking.

Meanwhile, Cornell's disclosure also calls for the remainder of the limb, which is not supported by the rigid support to be supported by the fabric sail support, to create a hydrostatic weight bearing support of the entire limb and its tissue. Conversely, in our disclosure, we may purposely maintaining open areas to allow the tissue to expand out as needed, so that we can compress the medial/lateral dimension, drawing the long bone into proximity with the anchor stabilizer.

The notion of using a rigid J-shaped support, as Cornell discloses, has been used in the prosthetics field for many years. Between 1999 and 2006 various tests were conducted at Sabolich Prosthetics to cut down the frame's trimlines to more a micro-frame design, resembling the Hi-Fi socket in look, though not necessarily fully in function with aggressive compression zones in the same way as Alley has demonstrated. These clinical fitting experiments, as well as the Hi-Fi sockets design, have demonstrated that the force coupling within a transfemoral socket can be achieved through a micro-frame structure, versus a fully encapsulated frame. Various tests were conducted using a J-shaped main frame, with a compliant silicon material encircling the limb that would be connected to the J-shaped frame. We found that the limb was able to remain stable in such a setup. Distal cups trimmed out at the distal end of the socket, and J shaped trimlines have been common in various socket shapes for many years (Schuch, Michael, Transfemoral Amputation: Prosthetic Management, Atlas of Limb Prosthetics 20B).

Other prosthetic component manufacturers provide components, such as gel liners, for use in conventional sockets, which do not significantly depart from conventional socket design. The Ossur Seal-In V liner for instance is a flexible themoplastic/silicon sock that rolls over the amputee's residual limb, which then fits into the conventional socket. The sealing rings of this design offer a better method of suspending the prosthesis than predecessor designs, by forming a suction sealing effect toward the distal end of the socket. While the liner does provide cushion for the user, and the suspension capabilities of this design work very well, it is but an iteration of conventional socket approaches, and fails to truly accommodate for volume changes in the residual limb.

Additionally, unlike Alley, Cornell, or Hurley's disclosure, our design does not require a proximal brim as is commonly used. Unlike any other interface design, we can effectively have a brimless socket interface, since this design is the only one which does not truly encapsulate the limb with a structure about the circumference of the entire limb. Instead, this disclosure may have floating elements, which may be modularly and adjustably tightened against the limb.

Still further, the other socket designs including Alley, Hurley, and Cornell, all utilize distal contouring of the limb within the socket, and as such bear a portion of the weight distally. Likewise, any weight bearing that is bore circumferentially around the limb extends directly to the distal attachment area. This invention however may utilize a non-weight-bearing distal end, and any force through the body of the limb may be bore through the stabilizing unit alone to the distal attachment area. By doing such, the size of the interface can be modularly adjusted circumferentially in real-time by the end user.

Any contouring of the interface about the distal end of the limb may come through compliant materials, versus rigid structure, as used by Alley, Hurley, and Cornell. The distal end of this invention may use more of a hammock-type fit with the contouring of the distal end to be modularly adjusted to the user, through compliant materials that match to the user, versus the user having to match to a pre-formed shape on the distal end of a conventional socket. Spanning fabric to create a distal end, if one is used, ensures comfort, and that there is not too much force applied in that area.

An open distal end, or a modularly adjustable distal end through compliant materials allows for open wounds on the distal end of the limb to be un-enclosed, and to promote healing. As such, this invention could be applied to a new amputee, quickly after the amputation, or to a user who needs to have their distal end de-weighted for healing purposes. An open air design makes for the skin environment to be significantly healthier, versus the conventional hostile interface environment of conventional sockets that encapsulate the limb in a hot, moist environment.

Even if weight is applied to the distal end of this invention, it is estimated that a relatively small amount may be in contact there, with a predominant amount, likely above 90% to be applied through the stabilizing unit, and its opposing force coupling means.

Likewise, this invention could be applied in developing nations, where there is a need for a modular prosthetic design that would not require time consuming an expensive custom fabrication processes, materials, and equipment. Through using off-the-shelf modular kit components, prosthetics can now be fit, either locally or in developing nations, inexpensively. And, since this invention offers so much modularity to fit various users, and fit with them with increased comfort and control, the end users life is enhanced. Each of the elements of this disclosure can be offered in a kit set, including the connectors, stabilizing unit and force distribution anchors, etc, to allow for quick and accurate fitting of prosthetics.

### SUMMARY OF THE INVENTION

The present invention relates generally to a new and improved prosthetic interface design. In particular, the present invention is a new and improved method of providing control and comfort within a prosthetic device.

In this respect, before explaining at least one embodiment of the invention in detail, it is to be understood that the invention is not limited in this application to the details of construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore that the claims be regarded as including such equivalent constructions insofar as they do not depart from the spirit and scope of the present invention.

Accordingly, titles, headings, chapters name, classifications and overall segmentation of the application in general should not be construed as limiting. Such are provided for overall readability and not necessarily as literally defining text or material associated therewith.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new and improved method of fitting prosthetics to those with limb loss.

It is a further object of the present invention to provide a prosthetic interface that is simpler and more consistent to fit to the user.

It is a further object of the present invention to provide a prosthetic interface whose fit is measurable, quantifiable, and repeatable.

It is a further object of the present invention to provide a prosthetic interface that is user adjustable.

It is a further object of the present invention to provide a prosthetic interface that is more breathable.

It is a further object of the present invention to provide a prosthetic interface that is lower profile under clothing.

It is a further object of the present invention to provide a prosthetic interface that is lighter in weight.

It is a further object of the present invention to provide a prosthetic interface that is modular and repairable.

It is a further object of the present invention to provide a prosthetic interface that can be fabricated less expensively and quicker.

It is a further object of the present invention to provide a prosthetic interface that uses compliant structures versus rigid or semi-rigid structures.

It is a further object of the present invention to provide a prosthetic interface that truly accommodates for volume and shape changes of the dynamic underlying body.

It is a further object of the present invention to provide a prosthetic interface that provides gradual transitions of forces at its trim lines.

It is a further object of the present invention to provide a prosthetic interface that does not encapsulate the limb in the same manner as conventional designs.

It is a further object of the present invention to provide a prosthetic interface that captures the lost biomechanical and neuromuscular connection between the limb and the user.

It is a further object of the present invention to provide a prosthetic interface to better control underlying bone position within the socket.

Another object of the present invention is to provide a new and improved system which provides some of the advantages of the prior art, while simultaneously overcoming some of the disadvantages normally associated therewith.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference would be had to the accompanying drawings and descriptive matter in which there are illustrated preferred embodiments of the invention.

### BRIEF DESCRIPTION OF THE PICTORIAL ILLUSTRATIONS, GRAPHS, DRAWINGS, AND APPENDICES

The invention will be better understood and objects other than those set forth above will become apparent when consideration is given to the following detailed description thereof. Such description makes reference to the annexed pictorial illustrations, graphs, drawings, and appendices.

FIG. 1A generally illustrates an embodiment of a transfemoral socket interface, viewed from a perspective angle.

FIG. 1B generally illustrates an embodiment of a transfemoral socket interface, viewed from a perspective angle.

FIG. 1C generally illustrates an embodiment of a transfemoral socket interface, viewed from a perspective angle.

FIG. 1D generally illustrates an embodiment of an attachment means connected to an embodiment of a force distribution stabilizer, viewed from a perspective angle.

FIG. 1E generally illustrates an embodiment of a transfemoral socket interface, viewed from a perspective angle, using compliant force distribution stabilizer structure with other compliant material spanned there between.

FIG. 1F generally illustrates an embodiment of a transfemoral socket interface, viewed from a perspective angle, using a less compliant force distribution stabilizer structure with other compliant material spanned there between.

FIG. 1G generally illustrates an embodiment of a transfemoral socket interface, viewed from a perspective angle, using a form holding structure spanned between force distribution stabilizers.

FIG. 1H generally illustrates an embodiment of a transfemoral socket interface, viewed from a perspective angle.

FIG. 1I generally represents possible cross sections of a proximal top-down view of an embodiment of the stabilizing unit 102, as may be affixed to the medial or lateral aspect of the transfemoral limb.

FIG. 2A generally illustrates an embodiment of a compliant structure as may be used for the gluteal fold area.

FIG. 2B generally illustrates an embodiment of a compliant structure as may be used for the gluteal fold area.

FIG. 3 generally illustrates another embodiment of a transfemoral socket interface, viewed from a perspective angle.

FIG. 4A generally illustrates another embodiment of a transfemoral socket interface, viewed from a perspective angle.

FIG. 4B generally illustrates another embodiment of a transfemoral socket interface, viewed from a perspective angle.

FIG. 5 illustrates an embodiment of the prior art, using an encapusulated socket interface.

FIG. 6A generally illustrates another embodiment of a transfemoral socket interface, viewed from a perspective angle.

FIG. 6B generally illustrates a close up view of an embodiment for a distal femoral stabilizing unit.

FIG. 7 illustrates the human anatomy, and the desired femoral angle in particular.

FIG. 8 illustrates various embodiments of the evolution of transfemoral socket interface designs.

FIG. 9 illustrates the human anatomy as it fits within one embodiment of conventional socket interface designs.

FIG. 10 illustrates another embodiment of a transfemoral socket interface, viewed from the perspective angle.

FIG. 11 illustrates the benefits of distributing forces through using compliant materials.

FIG. 12A illustrates another embodiment of an interface, viewed from the perspective angle.

FIG. 12B illustrates another embodiment of a transfemoral interface, viewed from the perspective angle.

FIG. 12C illustrates another embodiment of an interface, viewed from the perspective angle, for use in upper extremity.

FIG. 12D illustrates another embodiment of a prosthetic interface, viewed from the perspective angle.

FIG. 13 generally illustrates an embodiment of a transfemoral socket interface, viewed from a perspective angle, being worn by a user.

FIG. 14 generally illustrates an embodiment of a transfemoral socket interface, viewed from a perspective angle, being worn by a user.

FIG. 15 generally illustrates an embodiment of a transfemoral socket interface, viewed from a perspective angle.

FIG. 16 is a perspective view of a distal attachment embodiment.

### DETAILED DESCRIPTION OF THE PREFERRED EMBODIMENTS

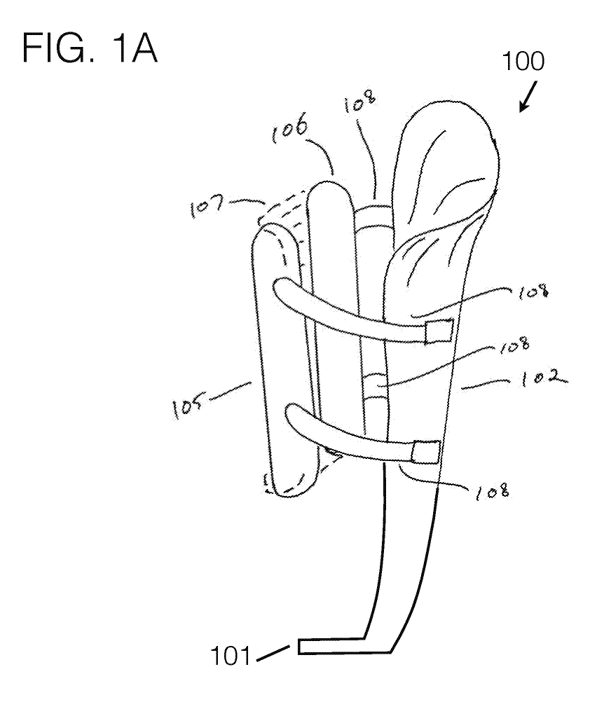

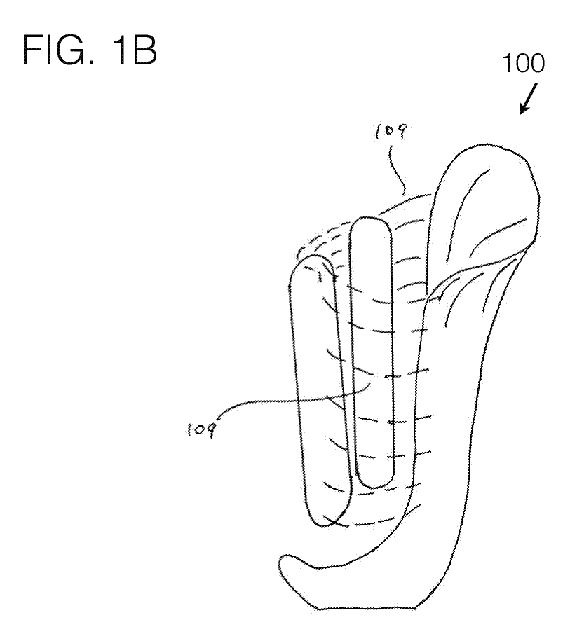

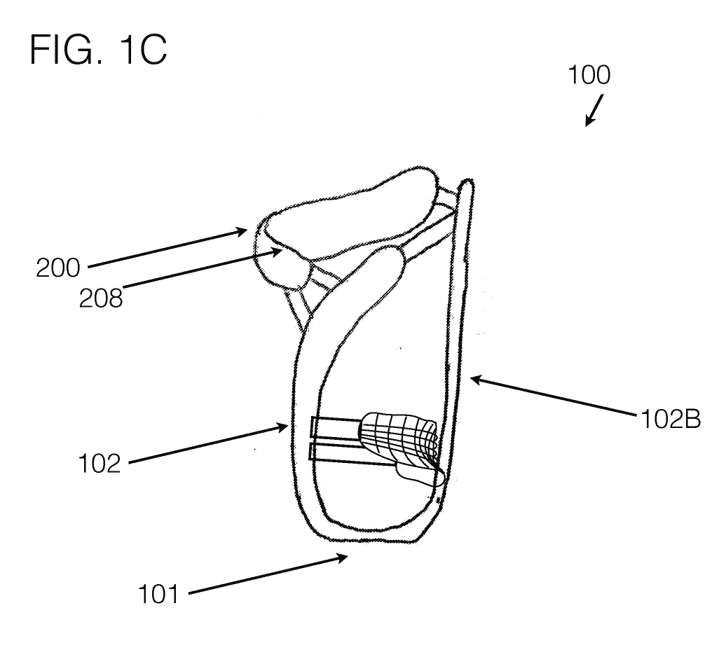

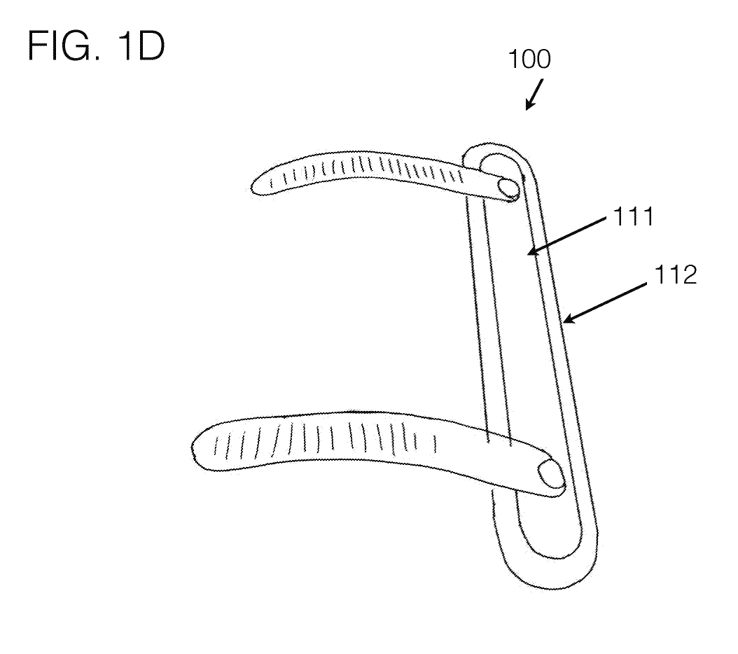

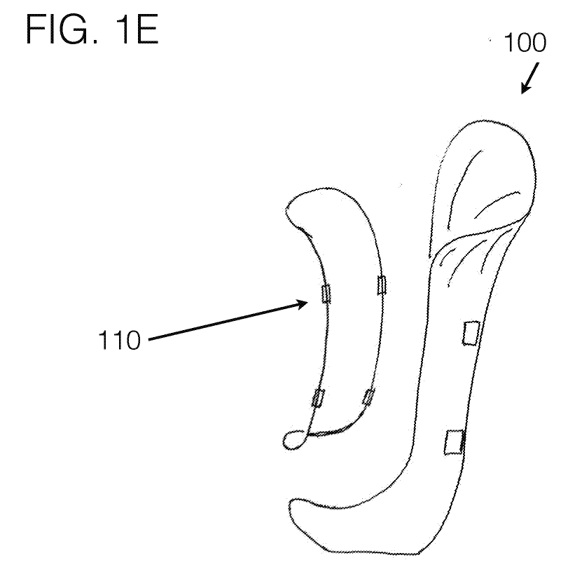

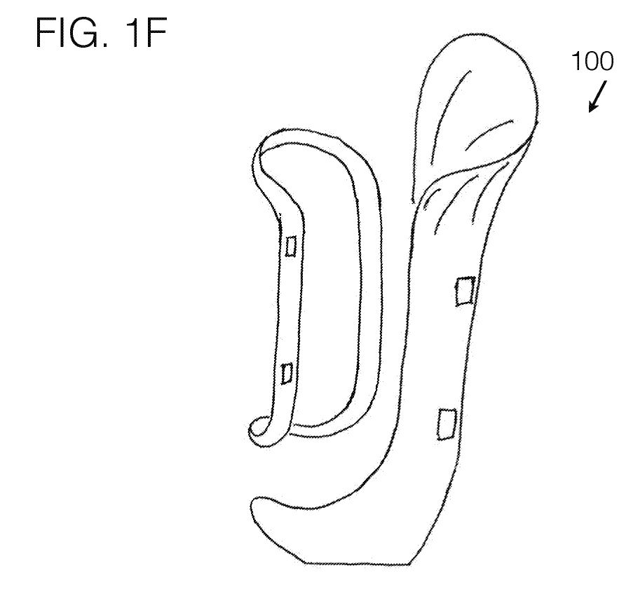

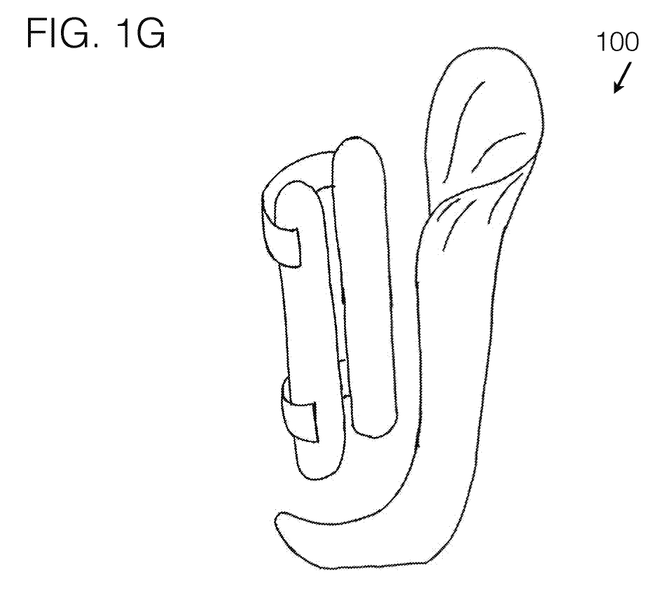

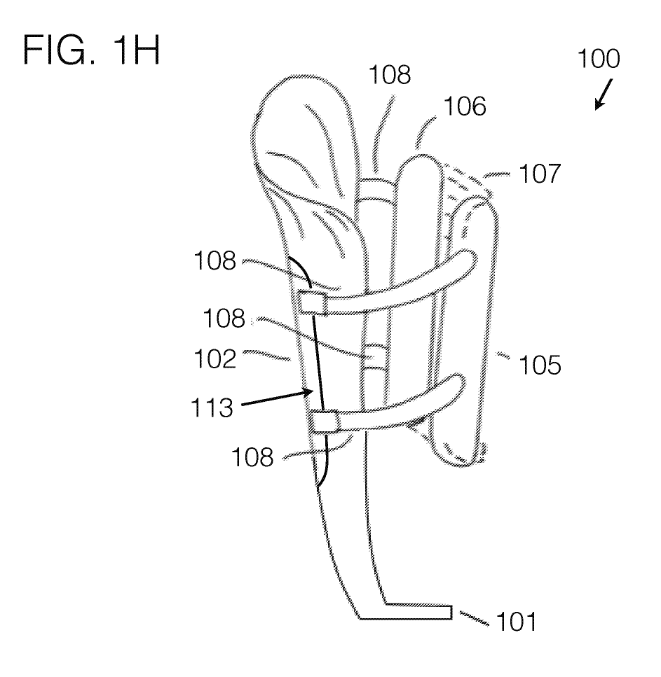

Referring now to the drawings, wherein like reference numerals designate corresponding structure throughout the views, and referring in particular to FIG. 1A, reference numeral 100 generally refers to a new and improved compliant based transfemoral level prosthetic socket apparatus, assembly and/or system, hereinafter referred to generally and collectively as invention 100.

Of note, invention 100 may be generally shown by example in a configuration for an individual missing a right or left leg or portion thereof at a knee disarticulation or transfemoral level. It is understood that such configuration is for example purposes only and that such should not be considered limiting and a left or right side configuration is also considered. It is further understood that invention 100 may be used where the level of amputation may dictate a different configuration than transfemoral or knee disarticulation level, such as but not limited to transtibial, transradial, transhumeral, or other levels either prosthetically, orthotically, or with exoskeletal robotics—all of which may be considered as human/machine connectivity. The terms should not be considered limiting the invention nor the general shape and configuration depicted in the drawings. Invention 100 may encompass many embodiments, as generally illustrated in the various figures, and should not be considered limiting where any particular figure depicts one embodiment of invention 100, as there are various elements, embodiments, and user specific requirements.

In a preferred construction, there may be a distal attachment area 101 for mounting other prosthetic components to, such as but not limited to knees, feet, stubbers, connectors, or other conventionally used components which are used distal to a socket apparatus. The particular attachment means may be any conventionally used means, including plates, screws, gunk, and others.

Extended from the general attachment area 101 may be a stabilizing unit(s) 102. The stabilizing unit 102 may be affixedly connected to the general attachment area, or may utilize elements floating in relation thereto. The particular contouring of the stabilizing unit 102 may be formed in any number of orientations and trim line cutouts, including various widths, heights, contouring, shape, attachment means, and other such elements. The stabilizing unit 102 may extend along any particular side of the limb, including but not limited to along the medial side, lateral side, anterior side, posterior side, or at an angle from one side distally, to a different side proximally.

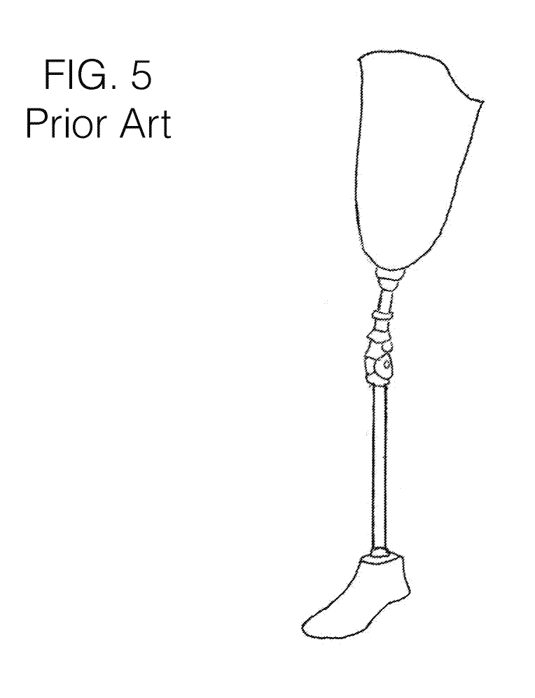

Conventional transfemoral interfaces typically largely circumferentially wrap around the limb, and provide a rigid support under the tuberosity area, as illustrated in FIG. 5. With invention 100 however, in a preferred embodiment, the stabilizing unit 102 may generally extend up the medial aspect of the limb near or between the quadriceps and hamstring muscle groups, or up the lateral aspect of the limb near or between the hamstring muscle group and the quadriceps muscle group, or along the anterior aspect of the limb near or between the hamstrings muscle group and adductor muscle group. The anatomical contouring between those groups may allow for a slight twist to the stabilizing unit 102 as it moves proximally up the limb, as is illustrated in FIG. 1C.

In a preferred embodiment, the stabilizing unit 102 may be relatively rigid to allow for support of the forces imposed through the device. The width of the stabilizing unit 102 may be tailored to individual users needs, and those illustrated in the figures should not be considered limiting.

In a preferred embodiment, there may be an additional stabilizing unit 102B, which may generally run proximally from the attachment area up the relatively opposing aspect of the limb. Additional stabilizing units may be used, and should not be considered limiting. While this element may not be required to achieve the desired outcomes, it may provide for added stability. In such an example, this stabilizing unit may have a similar rigidity as the medial stabilizing unit 102. Each stabilizing unit may generally contour according to the shape of the underlying limb, or may be relatively generic in shape, contouring to a generic limb. The particular placement, shape, rigidity, number, material, and other characteristics of such a stabilizing unit may be modified on a case-by-case basis according to the particular users needs.

While the material selection may allow for rigidity of the stabilizing units, because of their inherent shapes they may exhibit somewhat of flexibility in certain directions. To create a solid enough structure for supporting the user through the prosthetic interface, connector means may be used to attach either a stabilizing unit to itself, or to attach two or more stabilizing units to each other. This may be accomplished through using compliant members.

Instead of using an encapsulated socket as in conventional fitting approaches, the invention 100 may use fabric based or compliant based members to encapsulate portions of the limb, or may encapsulate a significant portion of the limb.

Referring specifically to FIG. 1A, stabilizing unit 102 may generally contour to a limb. In such an example, it may as well incorporate other elements such a padding or foam to help further contour to the specific underlying anatomy of a user. Stabilizing unit 102 may as well incorporate modular elements to modify its height, angle, length, or other adjustable aspects.

In general, stabilizing unit 102 may offer general or specific contouring for the ischial seat toward its proximal end, as in the use-case of it running along the general medial aspect of a transfemoral limb, or may utilize a sub-ischial design. It may as well wrap around the distal aspect of a residual limb, as illustrated in section 104, whereby a relatively small area is encapsulated to seat the residual limb into. Or, the distal area 104 may offer a larger area where the distal aspect of the residual limb may be encapsulated.

In a preferred embodiment, the predominant amount of force may be taken proximal to the distal end of the limb, and the distal end of the limb may have a relatively small amount of total force, or even no force, as illustrated in FIG. 1A. In such a case, the majority of the loading force through the system may be along the stabilizing unit, proximal to the distal end. Where minimal force may be encapsulated within the distal aspect of the limb, such an area may extend for a portion up the socket interface length, and may resemble the lower portion of a conventional socket interface in shape. Likewise, distal area 104 may extend the full length up the socket interface shape for a flexible inner socket as is traditionally used for such level of amputation, and may be able to be utilized on an existing flexible inner socket interface. Even further, distal end 104 may utilize compliant materials to encapsulate the distal end of the residual limb, thereby hammocking the distal end of the limb in a compliant material, which may include a fabric or other compliant materials.

FIG. 1I generally represents various possible cross sections of a proximal top-down view of an embodiment of the stabilizing unit 102, as may be affixed to the medial or lateral aspect of the transfemoral limb for instance. Such stabilizing unit may utilize one or more components of the stabilizing unit to be affixed to the distal attachment area 101, which may be modularly adjustable or not modularly adjustable in angulation, length, or position, or other orientations as may be desirable. Extended from the distal attachment area may be structural elements, which may include tubes 114, poles, struts 115, padding 116, or other such pieces, including any combination thereof, or may be custom fabricated as a single or multiple pieces, any combination of which used individually or together which may have enough structural integrity to support the necessary forces to generally support the user. Such pieces as a unit may help hold orientation about the limb, and may generally contour with respect to the long bone 117, generally causing a portion of the stabilizing unit to reside anterior to the long bone (anterior lateral, or anterior medial as the case may be), and one portion of the stabilizing unit to reside posterior to the long bone (posterior lateral, or posterior medial as the case may be). By doing so, the long bone may generally be held in a certain orientation with respect to the stabilizing unit, as the force distribution anchors may be tightened toward it, generally reducing the medial/lateral dimension of the interface. Stabilizing unit may be constructed of at least one independent component(s), with any such various components connected to the distal attachment area independently, and as a total working together as a unit. One example illustrated in FIG. 1I demonstrates the use of two independent components attached to the distal attachment area. Another example illustrates a single component attached to the distal attachment area. And a third illustration demonstrates a hole cut out in a single component, so that the distal end of the long bone may be relieved. Such illustration examples should not be considered limiting, as one or any other number of the examples may be used, or used in combination through the length of the stabilizing unit.

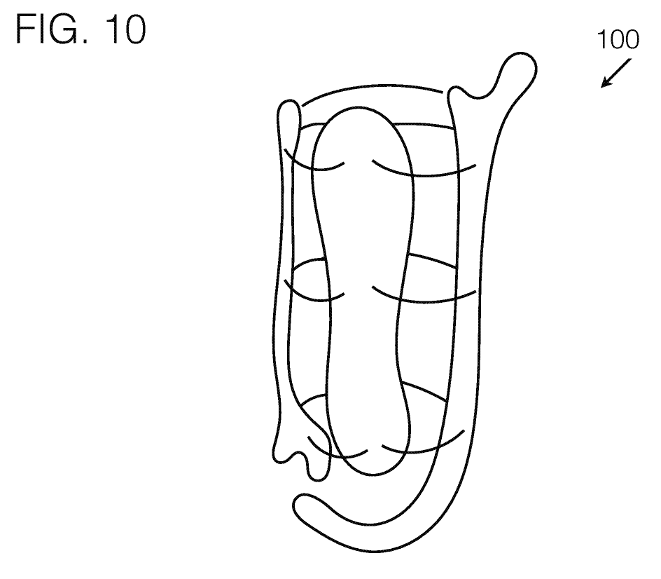

Force distribution anchor 105 and 106 may be positioned around the general opposing side of the limb, and may utilize a span of distance between their sub-components. The distance between force distribution anchor components 105 and 106 may be modularly adjustable, and may utilize a compliant material 107 between. The term compliant materials should not be considered limiting and in general may include a range of compliancy. For example, this may include materials such as fabric or mesh fabric, as well as materials like foam padding, fabric straps, Velcro, or thermoplastic ladder straps for typical ratchet mechanisms—each of which are compliant, and may offer appropriate levels of compliancy for different use-cases. Some use-cases require very flexible compliancy, whereas other use-cases may require a form-factor to be generally held, while still being able to be conforming under load. In general, the term compliant shall signify any material that is conforming to the body under the given load that is imposed on it, and which conforms an appropriate amount for the given use-case. Force distribution anchors 105 and or 106 may be fabricated of a relatively stiff material, or may be highly compliant. In a preferred embodiment, they may be stiff enough to hold their form with respect to the limb, and may be used as an anchor point for attachment means within the system. The force distribution anchors 105 and or 106 may be relatively narrow long shapes as illustrated in FIG. 1A or may offer other various shapes, such as but not limited to that which is illustrated in FIG. 10. Their specific shape should not be considered limiting, as they may be a modular component, or may be customized to fit a particular user, who may have particular shape dependent needs. Force distribution anchors 105 and or 106 may as well utilize markings to help a practitioner determine appropriate trim patterns or hole patterns for modularity. They may also offer spring response, so that during ambulation on the device, they may provide shock absorption for the user.

In another embodiment, the force distribution anchors 105 and or 106 may be fabricated from flexible materials, such as but not limited to wires, to manage the forces for their intended function.

Attached to the force distribution anchors 105 and or 106 may be adjustable or non-adjustable connectors 108 that may connect the force distribution anchors to the stabilizing unit. These connectors 108 may allow the force distribution anchors 105 and or 106 position to be modularly adjustable with respect to the stabilizing unit. There may be any number of connectors 108, and connector types that may be utilized, and the particular use of connectors in the figures should not be considered limiting. The connectors 108 may even utilize fabric spanned to accomplish the same.

In a preferred embodiment, the connectors 108 may incorporate areas which may maintain a certain curvature shape which may generally resemble the arc around a residual limb, whereas to help prevent the connectors 108 from roping across the limb. In such an example, the connectors 108 may utilize incorporated more rigid elements, which may as well provide some spring response during ambulation, or may be rigid enough to not allow spring response. In general, such features may help prevent the connectors 108 from digging into or roping into the limb as they arc around the curvature of the limb. Likewise, a broader material may be used to help spread the load across, thereby preventing them from digging into the limb.

FIG. 1B illustrates such an embodiment, where broad compliant fabric 109 or other compliant materials may be used to connect the force distribution anchors to the stabilizing unit. Such span of compliant material may be attached with any conventional attachment means, and such material may utilize connector means that may be modularly adjustable to allow for ease of tightening to a desired length.

FIG. 1D generally represents an embodiment of a force distribution anchor with connectors attachments on one side, in order to give a representation of how they may integrate within such an element. In such an example, there may be various materials used on within the force distribution anchor to allow for certain areas to be more rigid 111 and certain areas to be more flexible 112, to allow for an effective tapered transition of forces as it sits around the body. Additionally, as fabric or other compliant materials may be stretched from one stabilizing unit to another, and so forth, the fabric itself may create the soft transition from one structural element to another.

In addition, the connector means which may be used to connect either force distribution anchors with each other, or force distribution anchors to stabilizing unit may have semi-rigid elements or customizable elements to provide a set curvature. In doing so, it may help prevent the connector means from roping into the soft tissue as they curve around the limb. Further, the integration of other compliant materials such as fabric to span there between may be used to prevent roping, as it would effectively spread the forces over a broader surface area.

In between the force distribution anchors may be compliant fabric 107, which may or may not be modularly adjustable, to determine the span in between the force distribution anchors. Likewise, a rigid, semi-rigid, or other flexible means may be used to connect the force distribution anchors together, including forming the force distribution anchors together as a single continuous piece with like or dislike materials. It should therefore be understood the force distribution anchors may function as a unit, giving general opposing force to the stabilizing unit, and as such, may be considered a functioning single unit.

FIG. 1E generally represents an embodiment of how compliant fabric, fabric mesh, or other compliant materials may be spanned between a force distribution anchor system of one continuous piece. In such an example of embodiment FIG. 1E, the fabric may generally be bridged somewhat similar to a hammock between the force distribution anchor elements. Such assembly may utilize connection means to the stabilizing unit 102, or may use force distribution cabling run through such fabric to create a “paddle” of fabric. The perimeter outline shown in the figure may generally follow what becomes the curvature of the fabric paddle, as the force distribution cabling may be stretched there between in a certain configuration to cause the paddle to represent a pringle-shape, or other shapes may be utilized as well. The force distribution cabling may be compliant, though may cause the structure, which includes the compliant fabric stretched there between, to create a structural unit. Such structure may additionally include attachment means 110 to connect to the stabilizing unit 102. Additional fabric may be spanned between the paddle and the stabilizing unit 102. Additionally, other attachment means may be used to span to connect between as well.

FIG. 1F is similar to FIG. 1E, except that the force distribution anchors may be of a more structural nature formed as a continuous piece, or as a combination of multiple pieces configured to create a structure. FIG. 1E may represent a compliant continuous piece, or combination of pieces. Both examples may utilize fabric or other compliant material spanned in between to further spread out the load across the user's limb.

Still further FIG. 1G may represent an embodiment where the force distribution anchors may be connected together with a semi-rigid strut element, which may alternatively be semi-flexible, which may generally span away from the limb, so that as the force distribution anchors may be pulled toward the main stabilizing unit with their connection means (not shown in the figure), the force distribution anchors may press into the limb tissue. As such, there may also be fabric or other compliant material spanned between the force distribution anchors in addition, all of which may be modularly adjustable in their varying orientations, positions, and general effective contouring about the limb, to modify how they interact with the soft tissue of the limb.

Referring to FIG. 1C, and of which may generally be relevant to and embodied within other embodiments as well, on the proximal end of the interface, invention 100 may utilize a compliant structure to contour around the underlying anatomy, which may generally run from approximately near, at, or posterior to the adductor muscle group region toward the proximal end of the interface connecting at or near the stabilizing unit 102, and run generally around the posterior, or medial/posterior aspect of the limb toward the trochanter area of the lateral aspect of the upper thigh, which may attach to stabilizing unit 102B, or may continue further around the limb back to the adductor region connection point(s). In one embodiment, this element may simply connect the stabilizing unit to the posterior lateral force distribution anchor area, whereas it may generally be positioned near the gluteal fold region, to provide at least one of contouring, comfort, and control of the device.

FIG. 1H generally represents an embodiment where stabilizing unit 102 may generally extend from distal attachment means 101 proximally up the general lateral aspect of the limb, and whereas the force distribution anchor 105/106 may generally extend across the medial aspect of the limb. As such, the anterior force distribution anchor may generally sit near or between the quadriceps muscle group and the adductor muscle group, while the posterior force distribution anchor may generally sit near or between the adductor muscle group and the hamstring muscle group. In such an embodiment, the femur may generally be pulled laterally toward the stabilizing unit, and such stabilizing unit may exhibit a general angulation similar to the desired femoral angle.

Embodiment FIG. 1H may generally utilize a load bearing compliant member or structure 200 which the user may rest into during load bearing. Such unit may be incorporated within the force distribution anchor assembly or may be independent from such. By being compliant, such unit may provide increased comfort for the user, versus the traditional rigid ischial/ramus/tuberosity shelf found in conventional transfemoral sockets. As such, this element may function somewhat similar to the medial/posterior aspect of a rock climbing harness, in that some of the weight bearing of the unit may be bore in soft compliant materials, versus rigid structures. The lateral orientation of the stabilizing unit may allow the compliant member 200 generally be supported in the correct orientation with respect to the body.

This compliant member 200 may be utilized with stabilizing unit alone, or may incorporate force distribution anchors to assist in managing the direction and orientation of the forces through the system. It has been found clinically that the integration of the force distribution anchors within such an embodiment may provide added control and comfort.

In such an embodiment, the stabilizing unit extended up the general lateral aspect of the limb generally may make it more conducive for more of a generic shape to fit to a wide variety of limbs, versus having to be custom fabricated.

In an embodiment where stabilizing unit may extend up the lateral aspect of the limb, an opening 113 may exist in such stabilizing unit to allow for the long bone and/or tissue surrounding the long bone to fit within. As such, the distal end of the long bone may have space to press into a space where there is no rigid structure. This space may be spanned with no material, or may be spanned with compliant fabric to further control tissue flow. It is understood that such opening may be in any width, height, contouring, or shape as may be best suited for the particular patient, or for human anatomy as well. Stabilizing unit may exist in various subcomponents to allow for such opening to be created, including but not limited to disconnected anterior and posterior support sections, each of which may be connected to a distal and/or proximal end together, or to other such structure, including area 101. Such opening may also be used where the stabilizing unit resides along the medial aspect of the limb.

In general the term medial and lateral are in particular reference to a transfemoral use-case, and for other use-cases such as transtibial, transradial, transhumeral, or for orthotic applications, the particular orientation of the compression may best be utilized in an orientation other than medial/lateral, such as but not limited to anterior/posterior, and as such the general terminology should not be considered limiting, as the terms medial/lateral for the stabilizing unit and force distribution anchors general opposing force directions are for example purposes only for the transfemoral use-case, to allow one skilled in the art to better comprehend how they may relate with one another.

Amongst FIG. 1 general embodiments, force distribution anchors may generally float with respect to the stabilizing unit 102. By doing such, their circumferential position about the limb may be modularly controlled, allowing for full accommodation to the user's limb size and shape. Furthermore, if two force distribution anchor components are joined together as one unit, they may be positioned on either side of the long bone of the limb segment, allowing the more compliant material spanning in between, which may also be an area without material spanned in between, to be positioned over the long bone. By doing such, the force distribution anchors may effectively help lock the bone position such that the long bone is generally controlled during ambulation, through using the device. The compliant material, which may span between the force distribution anchors may allow for the sensitive distal end of such bone to be free of contact with any rigid or semi-rigid surface. One component of the force distribution anchor may generally reside on the anterior side of the long bone (anterior/medial, or anterior/lateral depending on orientation of the stabilizing unit being laterally or medially orientated), and one component of the force distribution anchor may generally reside on the posterior side of the long bone (posterior/medial, or posterior/lateral depending on the orientation of the stabilizing unit being laterally or medially oriented).

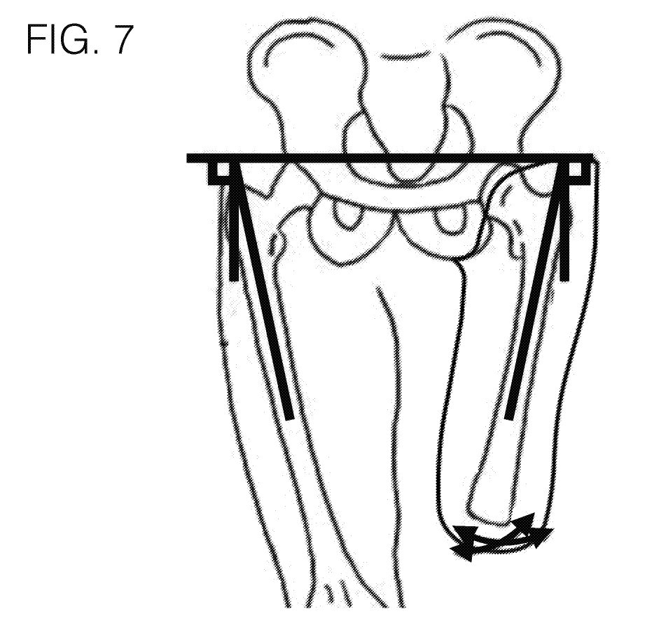

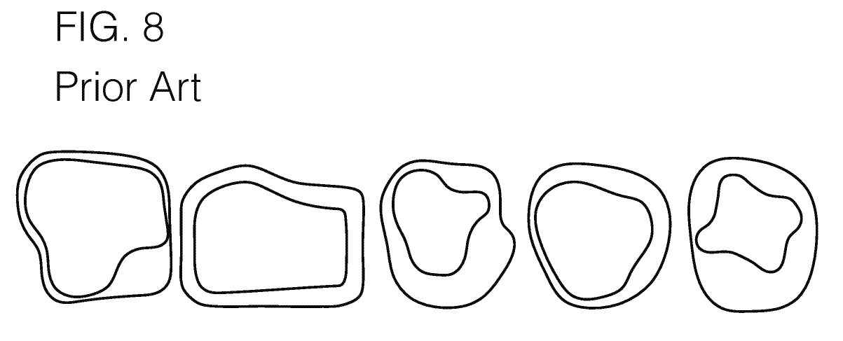

As the force distribution anchors may be tightened toward the stabilizing unit 102, it may generally shorten the medial/lateral dimension of the interface, as in the case of using this on a user with a transfemoral amputation for instance. In such a case, the long bone may be generally pulled toward the stabilizing unit, and maintained in such a position, as is referenced in FIG. 7 with the desired femoral angle. To accomplish this, the limb tissue may need to be displaced, in which the anterior and posterior dimensions may allow for the material to be displaced into, so that the medial/lateral aspect of the interface can be tightened to control the bone. The force distribution anchors may work in coordination with one another as effectively one unit locking the femur from the anterior and posterior sides, along with any material that may connect between the two, to control the position of the long bone.

It should be understood that while the invention is described in these configurations. Further, embodiments from FIG. 1A may be utilized on other levels including transhumeral, transradial, and transtibial levels with similar advantages as for a transfemoral level. Still further, embodiments illustrated in FIGS. 1A-1I may also be utilized in similar orthotics and exoskeletal robotics levels to control the underlying limb segments. The shapes of the stabilizing unit and force distribution anchors may embody many various configurations, and those illustrated and discussed should not be considered limiting. The general principles how such pieces may connect together, and how the pieces may work together to control the limb can be accomplished with a variety of configurations.

Referring to the horizontal attachment means of structure 200 as illustrated in FIG. 1C, the compliant structure 200 may generally be used to support load bearing of the user within the device. This element may be utilized in any of the embodiments, and generally may be spanned near or as the proximal posterior connector, running along the gluteal fold region. Instead of solely supporting the user's weight volumetrically through the whole limb as in conventional devices, a sizable amount of the vertical loading may be bore through such compliant member, and specifically it may run near the gluteal fold region to help accomplish the soft tissue and anatomical contouring as loading. Through using it in the configuration where it may extend further under the medial aspect of the limb as well, it may utilize ischial loading as well. This may function similar to how a rock climbing harness suspends its user, though may be accomplished here for use in prosthetics interfaces. This compliant member may further extend to or past the ischial area, so that such area may be supported by a compliant member, similar to how a rock climbing harness may function, instead of using a rigid or semi-rigid seat as in conventional socket interface designs.

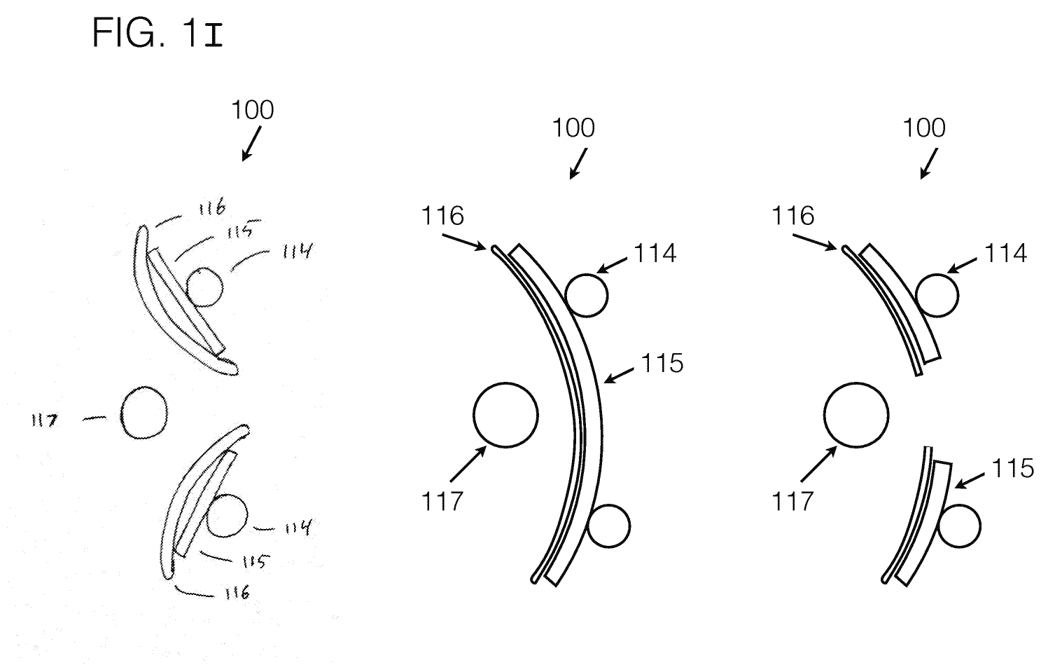

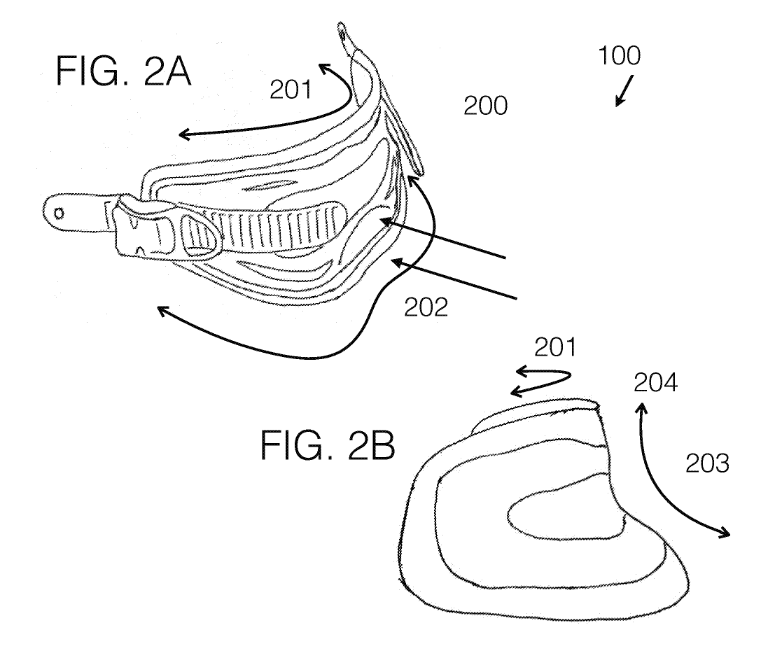

FIG. 2A and FIG. 2B generally shows a preferred embodiment of such a compliant structure 200, here specifically depicted as a gluteal stabilizer. The structure may encompass adjustable attachment means on its medial and lateral sides. These attachment means may be any commonly used in the industry, and those depicted should not be considered limiting, but may include user adjustable means, or non-user adjustable means, or a combination of both. The compliant nature of the compliant structure itself should, in a preferred embodiment, lend itself to a formed shape, while maintaining compliancy.

The attachment means may allow for adjustability in fit through tightening or loosening the compliant structure, changing the circumferential dimension of the interface. It may also be used to connect the stabilizing unit(s) which may provide added structural support of the interface unit.

It may encompass various materials of various degrees of compliancy to maintain such, including but not limited to fabric, foam, plastic, or other generally compliant materials. In general, the compliant structure may conform around or near the gluteal fold area of the body on the posterior side of the body. It may also offer a level of concavity or arc 203, which may help to contour into the soft tissue between the hamstrings and the buttocks.

The compliant structure 200, in a preferred embodiment, may have asymmetrical contouring as depicted in curvature 201 versus curvature 202, and curvature 204 versus curvature 205. In such an example, the curvature 201 may be notably different than that of curvature 202 to contour over various users differently. Some users may benefit from curvature 202 positioned on the distal aspect of the structure. Conversely, other users may benefit from the unit being positioned 180 degrees, with the curvature 201 positioned on the distal aspect of the strap. Having a reversible design may allow for better user contouring and success.

Likewise, asymmetrical contouring of arc 203 may benefit various users. Positioning the unit with the broader curvature of section 205 on its distal aspect may benefit those patients with larger soft tissue areas, which positioning the unit such that arc curvature 205 is positioned on its proximal side may benefit users with other body shapes.

The compliant materials of invention 100 may as well benefit from accessory elements such as integrated nanotechnology or other technologies to provide various characteristics that benefit the functional or user performance or experience with the device. These may include, but are not limited to, sensors, hygiene elements, antimicrobial elements, water repellency, or others.

This structure may as well integrate in purposed contouring to fit around the tuberosity and ramus areas of the body 208. This area may offer differing degrees of conformity or rigidity, in order to provide the necessary support for the user's needs.

There may be a generally similar compliant structure connecting the medial stabilizing unit 102 to lateral stabilizing unit 102B, as well as others as integrated. This may be used to help provide added structural stability of the interface unit, as well as provide sufficient comfort for the user on the proximal anterior aspect of the interface.

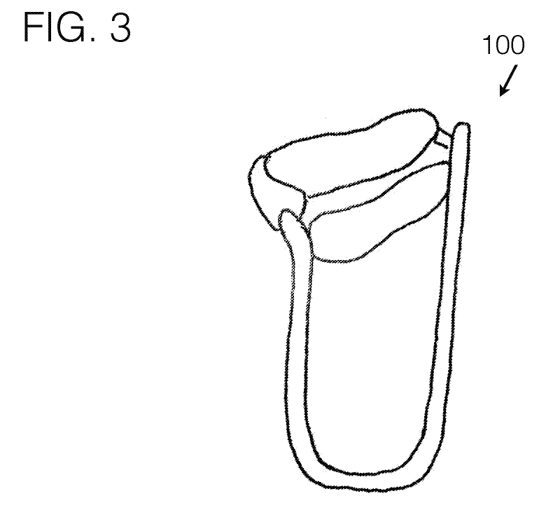

FIG. 3 generally illustrates an alternative embodiment of the invention 100 where the medial stabilizing unit may generally contour up the medial aspect of the limb, and may further contour around or near the tuberosity area, providing support through the stabilizing unit 102, versus directly through the compliant structure 200. There may also be another connector means running along the anterior connecting the various stabilizing units. Additional compliant material may be spanned in between across and around the general circumference of the limb, to help control tissue and connect to the stabilizing unit(s).

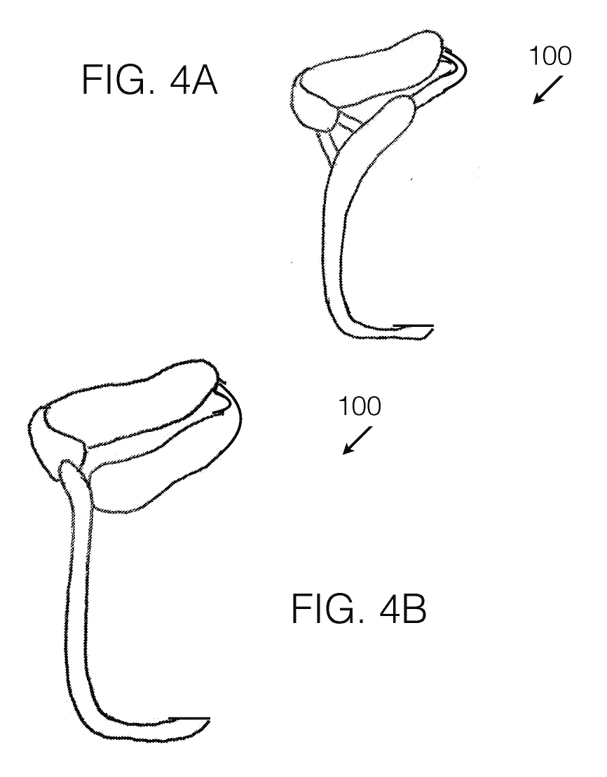

FIG. 4A and FIG. 4B generally illustrate similar configurations as shown in FIG. 1A and FIG. 3, although utilizing just a medial stabilizing unit 102, versus in combination with a lateral or other numbers of stabilizing units. In such an example, other compliant members may additionally be integrated to prevent user movement within the device in certain orientations of movements. One such example of such may be to connect the proximal lateral aspect of the proximal circumferential unit as show, to the distal lateral aspect of the anchor stabilizing unit to prevent the circumferential unit from migrating proximally. This can be controlled with a compliant strap, versus a rigid element. Further areas of the open areas may be spanned with other compliant materials, including but not limited to compliant fabric mesh. These other materials may be used to provide control of the tissue of the limb. The use of such circumferentially spanned fabric may help to encapsulate and control the limb tissue through stretching it tighter or looser in certain areas and directions about the soft tissue. The illustrations in FIG. 4A and FIG. 4B may as well utilize force distribution anchors as illustrated in other embodiments as well. These are not shown in these figures for simplification purposes, and therefore should not be considered limiting.

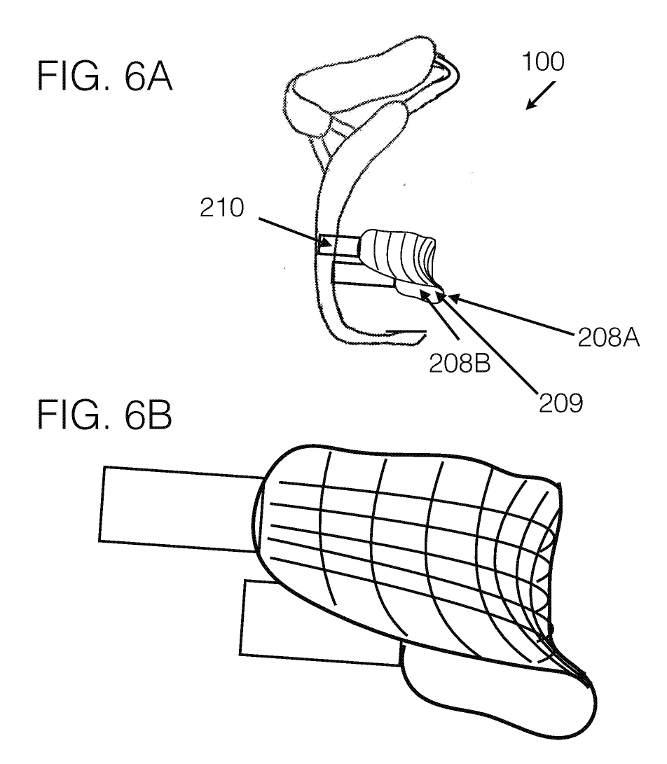

FIG. 6A generally illustrates the integration of a femoral stabilizer unit 600 integrated within the invention 100. In such an example, the general dynamic characteristics of the femoral stabilizer unit may be similar to the compliant structure 200, though may contour specifically around and proximal to the distal aspect of the femur bone. Its contouring may generally utilize 3-dimensional contouring sections to best contour around the underlying anatomy. It may offer lowered sections 208A and 208B to post either side of the femur bone, and a raised section 209 in between to allow the actual distal femur to not be impinged.

Adjustable connectors 210 may be used to allow for user adjustable tightening of the femoral stabilizer. The purpose of such a compliant structure may be used to not only post the sensitive distal aspect of the femur from the interface, but just as importantly, may help maintain femoral stability and femoral angle, thereby providing for a greater biomechanical stability of the femur within the interface. This will lead to greater control and stability for the user, as their bony structure within the residual limb will be more closely locked to the prosthetic movement. Such a femoral stabilizing unit may be used independently from, or in combination with force distribution anchor structure.

This femoral stabilizing unit structure may be attached at more than one location on each side, and may utilize other attachment sections (not shown in the figures) to provide increased positional stability of the unit with respect to the user's limb orientation. It should be understood that the illustration described in FIG. 6A and FIG. 6B may be functionally similar to that described in FIG. 1A-FIG. 1I, whereas the long bone may be more advantageously controlled by a connected, yet floating, element which may contour around the long bone in a way as to control its movement within the device.

Such an element may help maintain femoral stability within the design. FIG. 7 illustrates the femoral angle that should be maintained within a socket interface device. However, due to the cut end of the femur bone not being connected to the rest of the limb, the femur tends to move anterior/posterior, as well as medial/lateral during walking. This movement decreases stability and gait efficiency.

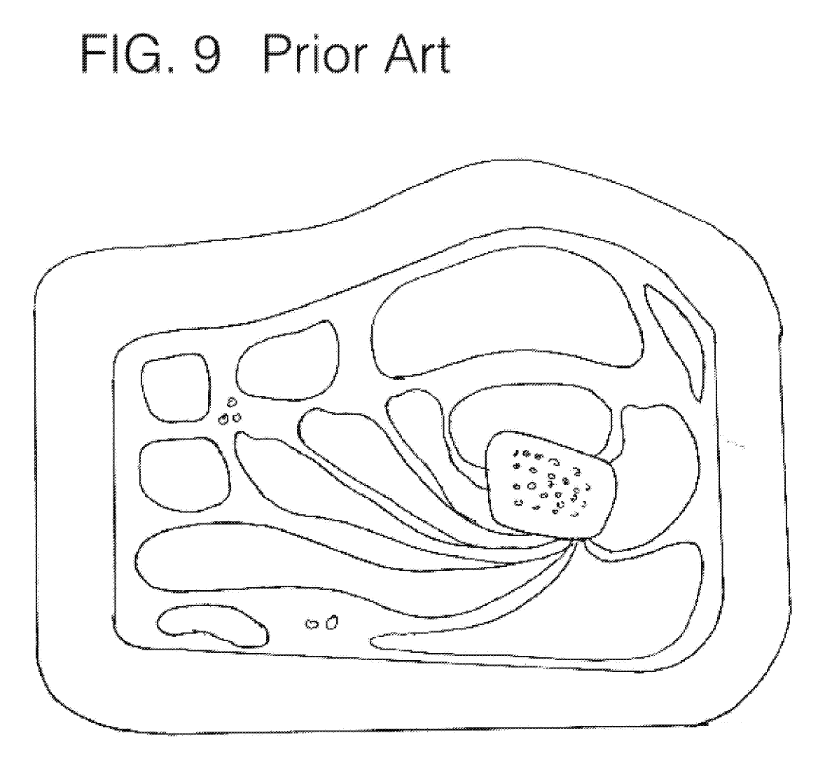

FIG. 8 demonstrates several of the evolutionary iterations of transfemoral socket design that have been used to help maintain femoral stability, as well as comfort and provide control of the limb. From left to right, these include: Plug fit socket, quad socket, Sabolich socket, MAS socket, Hi-Fi socket. Besides the Hi-Fi, each uses a hydrostatic fit, and all versions rely on enclosing the limb within an encapsulated thermoplastic socket, and none provide volume accommodation. Of these, only the Hi-Fi socket does an adequate job controlling the femur within the soft tissue. As one can see, the same anatomy of the human thigh can fit within many different shapes, and all of which may allow a user to effectively walk on their prosthetic device. This large variety of fitting methods is somewhat due to the compliant nature of the soft tissue of the thigh, however, each design offers different degrees of comfort and bony control. This may also suggest that the stabilizing unit as generally illustrated in FIG. 1A and others may be an off-the-shelf shape, which may be somewhat customizable to the user either through adjustment means or compliant materials incorporated within such as padding, to allow for a conforming and comfortable fit.

FIG. 9 generally shows how the muscles and bone fit within one version of a conventional socket design, in this case, showing an antiquated quad socket.

FIG. 10 shows an embodiment of invention 100 wherein the femoral stabilizing unit may be integrated within a vertical oriented force distribution anchor unit. Additionally, it may be integrated within a stabilizing unit or may be generally floating and connected to a main stabilizing unit via connectors. In such an example, there may be a main medial stabilizing unit, connected to floating anterior and/or lateral force distribution anchors. There may also be other numbers or locations, or contouring of such stabilizing units and force distribution anchors, and the illustrations should not be considered limiting.

In between the various force distribution anchors and stabilizing unit sections within the various embodiments may be a compliant fabric, which may include, but not limited to a mesh material. Such a material may offer breathability, coolness, lightweight, and durable design. In such an example, it may help encapsulate the limb tissue, providing for increased comfort and control. Even further, such compliant material may help post the distal femur and may allow the distal femur to contact only compliant fabric, versus rigid structure.

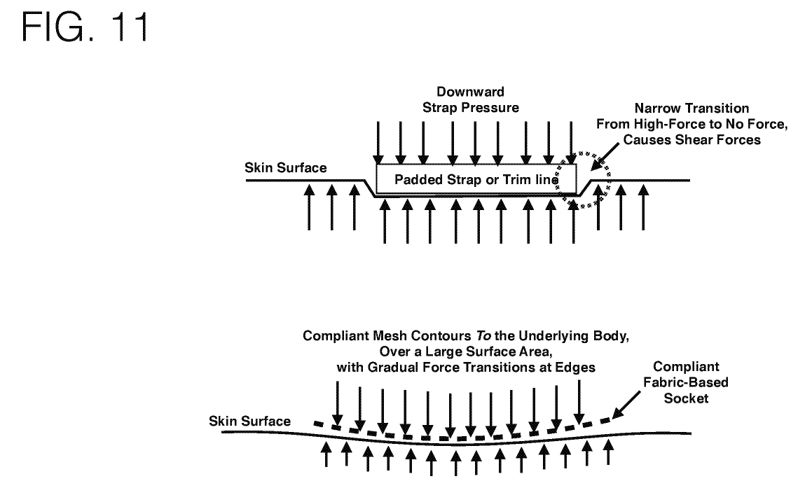

FIG. 11 demonstrates the benefit of integrating compliant fabric within the interface design, as it allows for a gradual transition from high forces to no forces. The fabric is not specifically illustrated in the figures for simplicity, though would reside between the various stabilizing elements, as may be integrated within the compliant fabric-based socket element. As a fabric may extend from a stabilizing element, the underlying tissue may be controlled at the transition points, allowing for gradual transitions of pressures, versus typical sharp transitions as is typically found in conventional fitting methods.

In addition to the socket contouring as depicted in the various illustrations, there may as well be a distal cup or distal socket area which the limb may reside (not shown in some of the illustrations). This may allow for any portion (from partial to full) of the limb to be encapsulated, allowing for suction or vacuum suspension to be achieved. This element may be fabricated with conventionally known methods and/or materials. Still further, the user may use a gel liner or other compressive sock or the like in conjunction with invention 100, which may allow for suspension, and tissue encapsulation and control as desired. The gel liner for example may be used independently or in conjunction with other flexible inner socket elements.

Invention 100 may be used in coordination with an existing socket design, or with a conventional flexible inner socket integrated, such that the force distribution anchor assembly may advantageously be used to improve the fit of an existing socket, as well as minimize the complexity of fitting a conventional socket, as invention 100 may help to improve modularity and adjustability of the fit around a user.

The compliant force distribution socket may encapsulate the limb with compliant materials, such as but not limited to fabric mesh, and may eliminate the non-breathable hot and heavy thermoplastic socket. It may utilize isolated regions of compliant, yet stabilizing zones, and a broad distribution of forces to support the limb and minimize point pressures.

Each may be connected with adjustable connectors to allow the user to tension the tightness to a preferred comfort. The stabilizing unit segments and the compliant fabric may be adjustable—allowing for the clinical fitting process to be modularly customizable to the user, as well as user-adjustable for preferred security and comfort.

The medially oriented stabilizing unit may key into the soft limb tissue generally near or between the hamstrings and adductor muscle groups, and a lateral oriented stabilizing unit may generally position near or between the hamstrings and quadriceps muscle groups, and an anterior stabilizing unit may generally position near or between the quadriceps and the adductor muscle groups. The various stabilizing units along with opposing force distribution anchors together may provide opposing forces, locking the limb in relation to the interface, and hence providing an improved link between the intended musculature and bony structure movement of the body with the movement of the prosthetic limb. The less the bone moves within the soft tissue, the better biomechanical and neuromuscular control will be achieved, reducing energy expenditure of ambulation.

As each of the stabilizing units and force distribution anchors may be separate, yet linked, there is an infinite amount of modularity and user adjustability, creating a fully customized fit, and ability to accommodate for residual limb volume change. The predominant surface area of the interface may be open, or mesh fabric, allowing for breathability and heat dissipation. It may, but not necessarily be, generally utilize conventional socket shape contouring, with the added benefit of adjustability between the various stabilizing unit segments.

This design may also allow for significantly lower trim lines at the proximal brim, and may not necessarily require specific brim elements as in conventional socket designs.

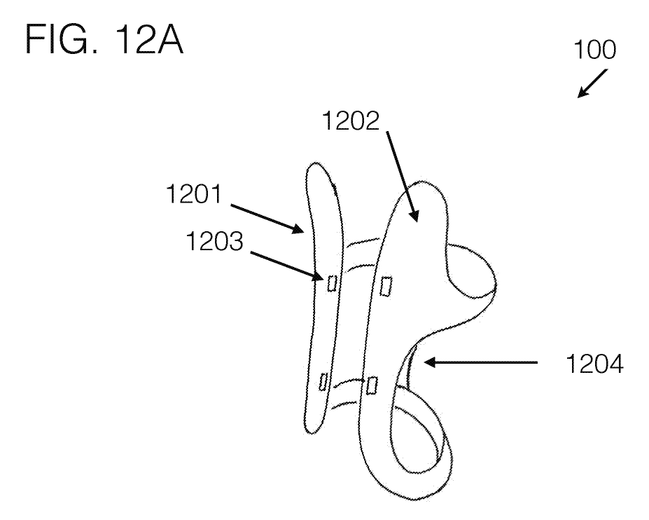

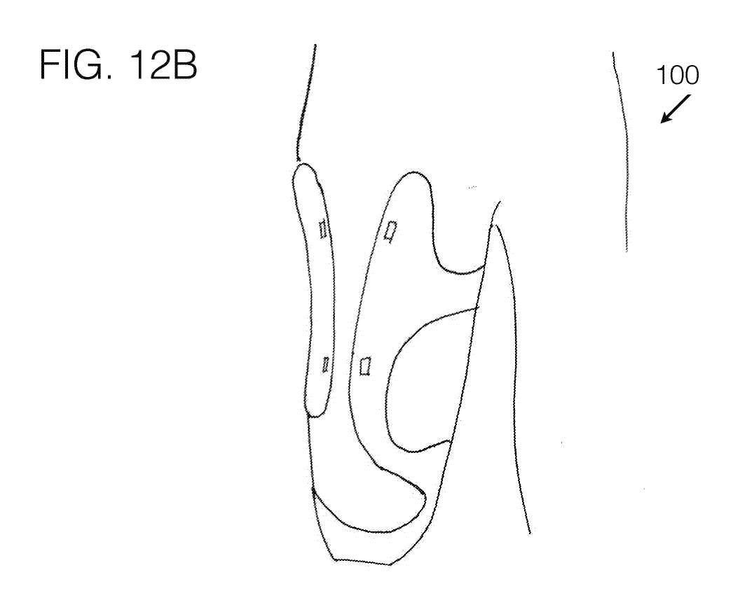

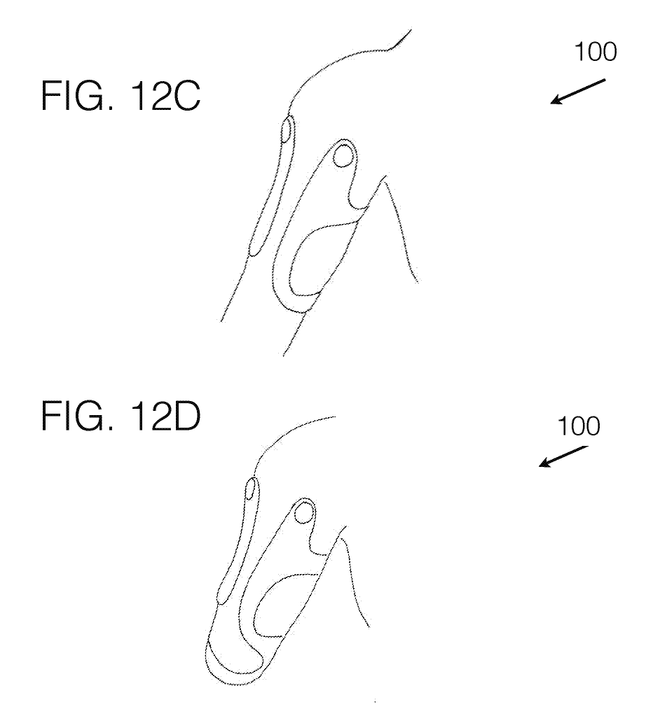

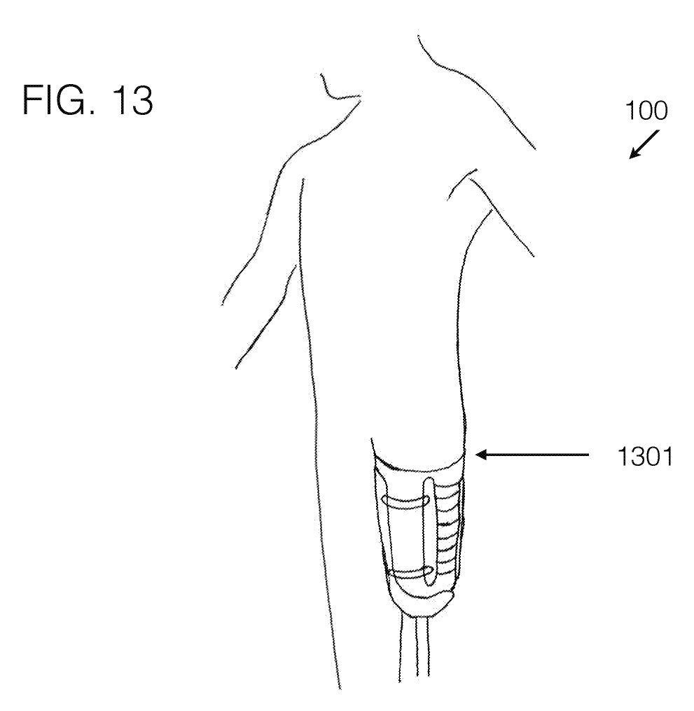

FIG. 12A generally illustrates an embodiment where the force distribution anchors may not float, but rather may be structurally connected. In such an example, the force distribution anchors 1201 and 1202 may generally run in an orientation along the long bone. In between them may be modularly adjustable connector means, which may also include compliant fabric. The connector means 1203 may allow the force distribution anchors to be pulled toward each other, tightening the interface about the limb. As fabric may be spanned or stretched in between the anchors, any long bone movement that may tend to press lateral, as in the case of a transfemoral amputee, may tend to push into the soft fabric, versus a rigid structure. In this embodiment, the interface may be fabricated to be slightly undersized for the user, thereby allowing it to have inherent compression about the limb, and inherent bony control. As the system may be tightened to the user, it may provide added compression. In general a tight medial/lateral compression may be used, to help control the bone, and allow the tissue to bulge out the other areas. Not illustrated in FIG. 12A, but is in FIG. 12B, at the distal end may be a connector means to attach to other typical components used along with such a device. Such an embodiment may also utilize a vertical anchor stabilizer 1204 along its general medial side, as illustrated for a transfemoral use-case, to prevent flexing of the structure. Additionally, padding or other compliant materials may be integrated to add comfort and conformability to the user.

FIG. 12B represents an embodiment as donned onto a transfemoral use-case. This illustration does not show the connector means, but it should be understood that they may be integrated into such a system.

FIG. 12C represents an embodiment as donned onto a transhumeral use-case, and as may be used for non-prosthetic man/machine interface connectivity.

FIG. 12D represents an embodiment as donned onto a transhumeral use-case.

In each such case, the connector means 1203 may be used to draw the force distribution anchors together, tightening the interface around the limb, and providing control of tissue and bony anatomy.

Such embodiments may also be used on other use-cases including transtibial, and transradial levels, and all such corresponding orthotics levels to control the limb segments. In any such orthotics use-case obviously an open end may be used, as the limb may extend past the end of the device. Furthermore, any such embodiments in this disclosure may be used in exoskeletal robotics, as they are merely advanced orthotics devices.

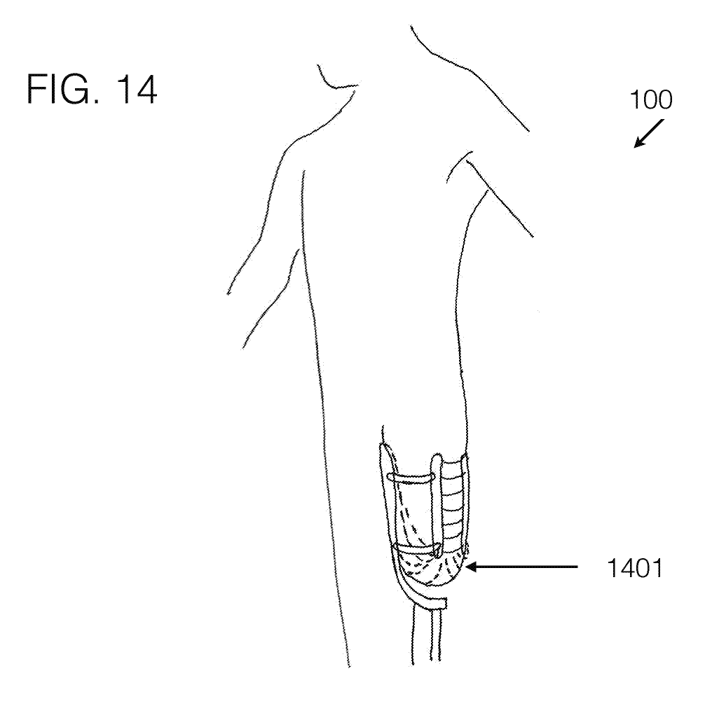

FIG. 13 represents invention 100 donned onto a transfemoral amputee use-case. This illustration shows how the distal end of the interface may be contoured to the distal of the residual limb. This illustration also shows the proximal end of a flexible inner socket, gel liner, or other compressive sock 1301 to control tissue, as may be worn with the device.

FIG. 14 represents invention 100 donned onto a transfemoral amputee use-case. This illustration shows how the distal end of the interface may purposely be in non-contact with the distal end of the residual limb structure. In such an example, the distal end of the limb may be suspended with compliant fabric 1401 to give a similar effect as a rigid structure's support, but with compliant means. This fabric section may be spanned from the stabilizing unit(s) and force distribution anchor(s) distally to their corresponding components proximally to provide an anchor for its attachment.

Likewise, such a system may be used for casting of a custom medial or lateral anchor, whereas the remaining elements of the invention 100 may be integrated to such a casting jig, in order to cause the plaster which may be wrapped around the residual limb to be specifically contoured to the underlying anatomy as the force distribution stabilizing units are tightened down to the user. Integrated fabric may assist in capturing the contouring of the distal end within the casting process. Similarly, the fabric may be spanned around much of the limb, creating a hammock containment of the limb within fabric, replicating or replacing a rigid interface that is conventionally used to hydrostatically manage the limb shape.

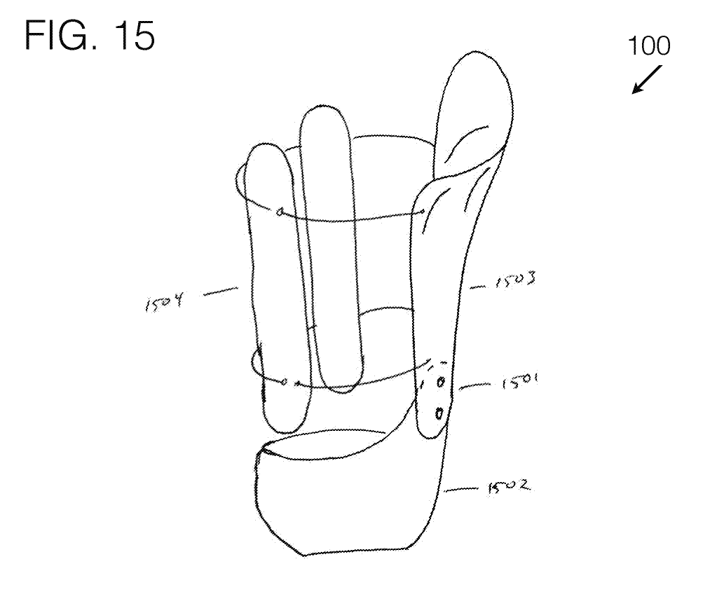

In an expanded modular embodiment the stabilizing unit may be of a fully modular or modular semi-customizable component, which may either attach directly to the limb components, or may attach to a customized distal connector, which may attach to the limb components, as illustrated in FIG. 15. It is also understood that other numbers of force distribution stabilizers or anchor stabilizers may be used, and their shapes, sizes, orientations, and configurations illustrated in the figures should not be considered limiting.

In such example, the attachment means 1501 may be any attachment method to known in the field, which may provide a structural attachment, and that in the figure is meant for illustrative purposes only. In such an example, the distal end of the socket 1502 may be customized by the clinical practitioner, and the modular stabilizing unit 1503 and force distribution anchors 1504 may be modularly connected within the system to make a complete interface, along with other sub-components. As described previously in FIG. 8, over the years there have been a number of socket design iterations which the human above knee limb can fit into, and all of which have radically different socket shapes. This tells us that the human thigh can fit within many different shapes, where the stabilizing unit may reside. As such, an off-the-shelf stabilizing unit may come in various sizes, or may offer other compliant means such as padding, and may generally offer a shape that resembles that of the underlying anatomy, in which it may contour around. Likewise, there may be any number of stabilizing units which may be modularly connected to a common customized distal attachment.

The embodiments represented may be custom fabricated, or may be pre-fabricated and sized to fit a variety of users, or may utilize a combination of both. Since they offer so much inherent modularity, a select few sizes will fit a variety of sizes of users. Additionally, conventional suspension systems available within the field may be used in conjunction with this design, including but not limited to distal cup or full length socket to provide vacuum or suction suspension, and which may be integrated into the invention.

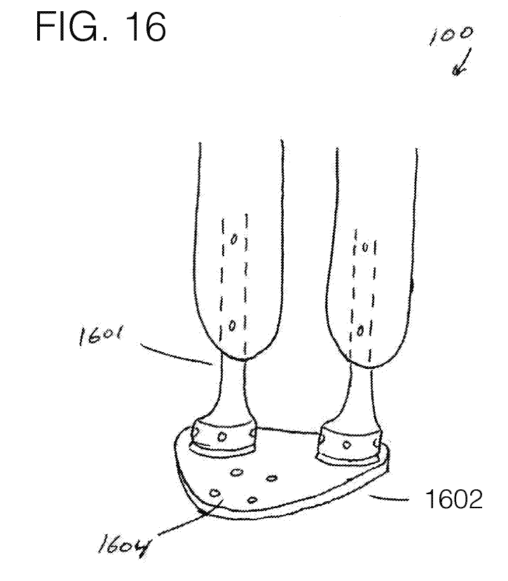

Invention 100 may utilize at least one, possibly two, or more, stabilizing unit(s) to be modularly connected to distal base. In one embodiment as illustrated in FIG. 16, stabilizing unit may have a rigid or semi-rigid section 1601 which may extend up the length of stabilizing unit, or may attach to a separate stabilizing unit section, which may resemble the force distribution anchors in general shape or characteristics. Such base of the stabilizing unit may be connected with adjustable base 1602. Such adjustable base may utilize any adjustment means known, and those illustrated should not be considered limiting. In one embodiment, adjustable base may utilize a male to female pyramid setup, where stabilizing unit section may be modularly adjusted in angulation. The opposing pyramid section may be modularly connected to a base plate such that the pyramid section may be adjusted in XY position on the base plate so that the desired femoral angle, weight line, and general biomechanics of alignment may be realized for the particular user. Connected to such base plate may be a mounting point for other prosthetic components 1604. Alternatively other attachment means may be used, and should not be considered limiting. Such base plate may be fabricated from metal, carbon fiber, or other such materials that may be sufficiently strong enough to support the users weight and forces. Holes may be placed in the material to modularly adjust the mounting positions of the various components.

It is therefore contemplated the invention may be an apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising: a medial segment having a top, a bottom, a length between said top and said bottom, and adapted to be positioned on said user said medial aspect on said circumference along said first length and wherein said bottom extends past said distal end and does not contact said surface on said distal end; a lateral segment having a top, a bottom, a length between said top and said bottom and adapted to be located on said user lateral aspect on said circumference along said second length; a first connector for connecting said medial segment to said lateral segment across said front aspect on said circumference; a second connector for connecting said medial segment to said lateral segment along said back aspect on said circumference; and a mounting point for an attachment located on said bottom of said medial segment; and wherein said first connector has an adjustable length for tightening and loosening said medial segment and said lateral segment around said residual leg circumference; wherein said second connector has an adjustable length for tightening and loosening said medial segment and said lateral segment around said residual leg circumference; and wherein said bottom of said medial segment extends past said distal end and does contact said surface on said distal end.

It is also contemplated that the invention may be an apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising: a lateral segment having a top, a bottom, a length between said top and said bottom, and adapted to be positioned on said user said lateral aspect on said circumference along said second length and wherein said bottom extends past said distal end and does not contact said surface on said distal end; a medial segment having a top, a bottom, a length between said top and said bottom and adapted to be located on said user medial aspect on said circumference along said first length; a first connector for connecting said medial segment to said lateral segment across said front aspect on said circumference; a second connector for connecting said medial segment to said lateral segment along said back aspect on said circumference; and a mounting point for an attachment located on said bottom of said lateral segment; wherein said first connector has an adjustable length for tightening and loosening said medial segment and said lateral segment around said residual leg circumference; wherein said second connector has an adjustable length for tightening and loosening said medial segment and said lateral segment around said residual leg circumference; and wherein said bottom of said medial segment extends past said distal end and does contact said surface on said distal end.

The invention still contemplates an apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising: a medial segment having a top, a bottom, a length between said top and said bottom, and adapted to be positioned on said user said medial aspect on said circumference along said first length and wherein said bottom extends past said distal end and does not contact said surface on said distal end; a first lateral segment having a top, a bottom, a length between said top and said bottom and adapted to be located on said user lateral aspect on said circumference along said second length; a second lateral segment having a top, a bottom, a length between said top and said bottom and adapted to be located on said user lateral aspect on said circumference along said second lengths wherein said first lateral segment and said second lateral segment are connected with a compliant material; a first connector for connecting said medial segment to said first lateral segment across said front aspect on said circumference; a second connector for connecting said medial segment to said second lateral segment along said back aspect on said circumference; and a mounting point for an attachment located on said bottom of said medial segment; wherein said first connector has an adjustable length for tightening and loosening said medial segment and said first lateral segment around said residual leg circumference; wherein said second connector has an adjustable length for tightening and loosening said medial segment and said second lateral segment around said residual leg circumference; wherein said bottom of said medial segment extends past said distal end and does contact said surface on said distal end; wherein said medial segment, said first lateral segment, and said second lateral segment are made from a rigid material; and wherein said compliant material is mesh.

It is further contemplated that the invention may be an apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising: a lateral segment having a top, a bottom, a length between said top and said bottom, and adapted to be positioned on said user said lateral aspect on said circumference along said first length and wherein said bottom extends past said distal end and does not contact said surface on said distal end; a first medial segment having a top, a bottom, a length between said top and said bottom and adapted to be located on said user medial aspect on said circumference along said second length; a second medial segment having a top, a bottom, a length between said top and said bottom and adapted to be located on said user medial aspect on said circumference along said second lengths wherein said first medial segment and said second medial segment are connected with a compliant material; a first connector for connecting said lateral segment to said first medial segment across said front aspect on said circumference; a second connector for connecting said lateral segment to said second medial segment along said back aspect on said circumference; and a mounting point for an attachment located on said bottom of said lateral segment; wherein said first connector has an adjustable length for tightening and loosening said lateral segment and said first medial segment around said residual leg circumference; wherein said second connector has an adjustable length for tightening and loosening said lateral segment and said second medial segment around said residual leg circumference; wherein said bottom of said lateral segment extends past said distal end and does contact said surface on said distal end; wherein said first medial segment, said second medial segment, and said lateral segment are made from a rigid material; and wherein said compliant material is mesh.

Changes may be made in the combinations, operations, and arrangements of the various parts and elements described herein without departing from the spirit and scope of the invention.

1. An apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising:
a medial segment having a top, a bottom, a length between said top and said bottom, and adapted to be positioned on said user said medial aspect on said circumference along said first length and wherein said bottom extends past said distal end and does not contact said surface on said distal end;
a lateral segment having a top, a bottom, a length between said top and said bottom and adapted to be located on said user lateral aspect on said circumference along said second length;
a first connector for connecting said medial segment to said lateral segment across said front aspect on said circumference;
a second connector for connecting said medial segment to said lateral segment along said back aspect on said circumference; and
a mounting point for an attachment located on said bottom of said medial segment.

2. The apparatus of claim 1 wherein said first connector has an adjustable length for tightening and loosening said medial segment and said lateral segment around said residual leg circumference.

3. The apparatus of claim 1 wherein said second connector has an adjustable length for tightening and loosening said medial segment and said lateral segment around said residual leg circumference.

4. The apparatus of claim 1 wherein said bottom of said medial segment extends past said distal end and does contact said surface on said distal end.

5. An apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising:
a lateral segment having a top, a bottom, a length between said top and said bottom, and adapted to be positioned on said user said lateral aspect on said circumference along said second length and wherein said bottom extends past said distal end and does not contact said surface on said distal end;
a medial segment having a top, a bottom, a length between said top and said bottom and adapted to be located on said user medial aspect on said circumference along said first length;
a first connector for connecting said medial segment to said lateral segment across said front aspect on said circumference;
a second connector for connecting said medial segment to said lateral segment along said back aspect on said circumference; and
a mounting point for an attachment located on said bottom of said lateral segment.

6. The apparatus of claim 5 wherein said first connector has an adjustable length for tightening and loosening said medial segment and said lateral segment around said residual leg circumference.

7. The apparatus of claim 5 wherein said second connector has an adjustable length for tightening and loosening said medial segment and said lateral segment around said residual leg circumference.

8. The apparatus of claim 5 wherein said bottom of said medial segment extends past said distal end and does contact said surface on said distal end.

9. An apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising:
a medial segment having a top, a bottom, a length between said top and said bottom, and adapted to be positioned on said user said medial aspect on said circumference along said first length and wherein said bottom extends past said distal end and does not contact said surface on said distal end;
a first lateral segment having a top, a bottom, a length between said top and said bottom and adapted to be located on said user lateral aspect on said circumference along said second length;
a second lateral segment having a top, a bottom, a length between said top and said bottom and adapted to be located on said user lateral aspect on said circumference along said second lengths wherein said first lateral segment and said second lateral segment are connected with a compliant material;
a first connector for connecting said medial segment to said first lateral segment across said front aspect on said circumference;
a second connector for connecting said medial segment to said second lateral segment along said back aspect on said circumference; and
a mounting point for an attachment located on said bottom of said medial segment.

10. The apparatus of claim 9 wherein said first connector has an adjustable length for tightening and loosening said medial segment and said first lateral segment around said residual leg circumference.

11. The apparatus of claim 9 wherein said second connector has an adjustable length for tightening and loosening said medial segment and said second lateral segment around said residual leg circumference.

12. The apparatus of claim 9 wherein said bottom of said medial segment extends past said distal end and does contact said surface on said distal end.

13. The apparatus of claim 9 wherein said medial segment, said first lateral segment, and said second lateral segment are made from a rigid material.

14. The apparatus of claim 9 wherein said compliant material is mesh.

15. An apparatus for attaching a transfemoral prosthetic to a user's residual leg wherein said residual leg has a circumference, a medial aspect on said circumference, a front aspect on said circumference, a lateral aspect on said circumference, a back aspect on said circumference, a distal end above where a knee would be and wherein said distal end has a surface, a proximal end at a hip area, a first length defined between said distal end and said proximal end on said medial aspect, a second length defined between said distal end and said proximal end on said lateral aspect, said apparatus comprising:
a lateral segment having a top, a bottom, a length between said top and said bottom, and adapted to be positioned on said user said lateral aspect on said circumference along said first length and wherein said bottom extends past said distal end and does not contact said surface on said distal end;
a first medial segment having a top, a bottom, a length between said top and said bottom and adapted to be located on said user medial aspect on said circumference along said second length;
a second medial segment having a top, a bottom, a length between said top and said bottom and adapted to be located on said user medial aspect on said circumference along said second lengths wherein said first medial segment and said second medial segment are connected with a compliant material;
a first connector for connecting said lateral segment to said first medial segment across said front aspect on said circumference;
a second connector for connecting said lateral segment to said second medial segment along said back aspect on said circumference; and
a mounting point for an attachment located on said bottom of said lateral segment.

16. The apparatus of claim 15 wherein said first connector has an adjustable length for tightening and loosening said lateral segment and said first medial segment around said residual leg circumference.

17. The apparatus of claim 15 wherein said second connector has an adjustable length for tightening and loosening said lateral segment and said second medial segment around said residual leg circumference.

18. The apparatus of claim 15 wherein said bottom of said lateral segment extends past said distal end and does contact said surface on said distal end.

19. The apparatus of claim 15 wherein said first medial segment, said second medial segment, and said lateral segment are made from a rigid material.

20. The apparatus of claim 15 wherein said compliant material is mesh.
